SEQUENCE LISTING (91 sequences)

SEQ ID NO: 1 - protein, 20 aa, synthetic construct
PSSRASSRASSRPRPDDLEI

SEQ ID NO: 2 - protein, 9 aa, synthetic construct
RPRPDDLEI

SEQ ID NO: 3 - protein, 9 aa, synthetic construct
RPRPDDLEV

SEQ ID NO: 4 - protein, 9 aa, synthetic construct
RPRPDDVPV

SEQ ID NO: 5 - protein, 9 aa, synthetic construct
KARSDDLSV

SEQ ID NO: 6 - DNA, 27 bp, synthetic construct
  source 1..27: mol_type = other DNA
agacctcggcctgatgacctggagatt

SEQ ID NO: 7 - protein, 16 aa, synthetic construct
RQPKIWFPNRRKPWKK

SEQ ID NO: 8 - protein, 36 aa, synthetic construct
RQPKIWFPNRRKPWKKPSSRASSRASSRPRPDDLEI

SEQ ID NO: 9 - protein, 25 aa, synthetic construct
RQPKIWFPNRRKPWKKRPRPDDLEI

SEQ ID NO: 10 - protein, 25 aa, synthetic construct
RQPKIWFPNRRKPWKKRPRPDDLEV

SEQ ID NO: 11 - protein, 25 aa, synthetic construct
RQPKIWFPNRRKPWKKRPRPDDVPV

SEQ ID NO: 12 - protein, 25 aa, synthetic construct
RQPKIWFPNRRKPWKKKARSDDLSV

SEQ ID NO: 13 - DNA, 74 bp, synthetic construct
  source 1..74: mol_type = other DNA
cggcagcccaagatctggttccccaaccggcggaagccctggaagaagcggcccggcccgacgacctggagatc

SEQ ID NO: 14 - protein, 11 aa, synthetic construct
GRKKRRQRPPQ

SEQ ID NO: 15 - protein, 16 aa, synthetic construct
RQIKIWFQNRRMKWKK

SEQ ID NO: 16 - protein, 16 aa, synthetic construct
RQIAIWFQNRRMKWAA

SEQ ID NO: 17 - protein, 9 aa, synthetic construct
RKKRRQRRR

SEQ ID NO: 18 - protein, 21 aa, synthetic construct
TRSSRAGLQFPVGRVHRLLRK

SEQ ID NO: 19 - protein, 26 aa, synthetic construct
GWTLNSAGYLLGKINKALAALAKKIL

SEQ ID NO: 20 - protein, 18 aa, synthetic construct
KLALKLALKALKAALKLA

SEQ ID NO: 21 - protein, 16 aa, synthetic construct
AAVALLPAVLLALLAP

SEQ ID NO: 22 - protein, 10 aa, synthetic construct
VPMLKPMLKE

SEQ ID NO: 23 - protein, 28 aa, synthetic construct
MANLGYWLLALFVTMWTDVGLCKKRPKP

SEQ ID NO: 24 - protein, 18 aa, synthetic construct
LLIILRRRIRKQAHAHSK

SEQ ID NO: 25 - protein, 21 aa, synthetic construct
KETWWETWWTEWSQPKKKRKV

SEQ ID NO: 26 - protein, 18 aa, synthetic construct
RGGRLSYSRRRFSTSTGR

SEQ ID NO: 27 - protein, 15 aa, synthetic construct
SDLWEMMMVSLACQY

SEQ ID NO: 28 - protein, 12 aa, synthetic construct
TSPLNIHNGQKL

SEQ ID NO: 29 - protein, 19 aa, synthetic construct
PSRASSRASSRPRPDDLEI

SEQ ID NO: 30 - protein, 20 aa, synthetic construct
GSNKSTASSKSPDPKNSVWI

SEQ ID NO: 31 - protein, 20 aa, synthetic construct
GSNKSSASSKSGDGKNSVWI

SEQ ID NO: 32 - protein, 21 aa, synthetic construct
GRASKASRASSGRARPEDLAI

SEQ ID NO: 33 - protein, 13 aa, synthetic construct
GSASSRDGKTVWI

SEQ ID NO: 34 - protein, 19 aa, synthetic construct
PRVSVPNFGRTQSSDSAYV

SEQ ID NO: 35 - protein, 19 aa, synthetic construct
PRMSMPNFGRTQSSDSAYV

SEQ ID NO: 36 - protein, 21 aa, synthetic construct
PRAGSEKGSASSRDGKTTVWI

SEQ ID NO: 37 - protein, 23 aa, synthetic construct
GYHSDKRRLSKASSKARSDDLSV

SEQ ID NO: 38 - protein, 19 aa, synthetic construct
PLSRLSKASSRARSDDLTV

SEQ ID NO: 39 - protein, 22 aa, synthetic construct
PNHVVSLTNNLIGRRVPTDLQI

SEQ ID NO: 40 - protein, 29 aa, synthetic construct
PSCVSSSAVLTTICSSDQVVPVGLSSFYM

SEQ ID NO: 41 - protein, 21 aa, synthetic construct
GRSSKASKSSGGRARAADLAI

SEQ ID NO: 42 - protein, 17 aa, synthetic construct
LCYLLIRYCSGKSKKPV

SEQ ID NO: 43 - protein, 18 aa, synthetic construct
GQKPPSRPSSSASKKQYV

SEQ ID NO: 44 - protein, 19 aa, synthetic construct
SSRASSRASSRPRPDDLEV

SEQ ID NO: 45 - protein, 9 aa, synthetic construct
RPKPDDLEI

SEQ ID NO: 46 - protein, 19 aa, synthetic construct
SSRASSRASSRPKPDDLEI

SEQ ID NO: 47 - protein, 9 aa, synthetic construct
RPKPDDLDI

SEQ ID NO: 48 - protein, 19 aa, synthetic construct
SSRASSRASSRPRPDDLDI

SEQ ID NO: 49 - protein, 19 aa, synthetic construct
SSRASTRASSRPRPDDLEI

SEQ ID NO: 50 - protein, 9 aa, synthetic construct
RPRPEDLEI

SEQ ID NO: 51 - protein, 19 aa, synthetic construct
SSRASSRASSRPRPEDLEI

SEQ ID NO: 52 - protein, 9 aa, synthetic construct
GDGKNSVWV

SEQ ID NO: 53 - protein, 23 aa, synthetic construct
SKAGSNKSTASSKSGDGKNSVWV

SEQ ID NO: 54 - protein, 18 aa, synthetic construct
GQKPPSRPSSSASKKLYV

SEQ ID NO: 55 - protein, 24 aa, synthetic construct
RQPKIWFPNRRKPWKIELDDPRPR

SEQ ID NO: 56 - protein, 20 aa, synthetic construct
GRKKRRQRPPQRPRPDDLEI

SEQ ID NO: 57 - protein, 25 aa, synthetic construct
RQIKIWFQNRRMKWKKRPRPDDLEI

SEQ ID NO: 58 - protein, 25 aa, synthetic construct
RQIAIWFQNRRMKWAARPRPDDLEI

SEQ ID NO: 59 - protein, 18 aa, synthetic construct
RKKRRQRRRRPRPDDLEI

SEQ ID NO: 60 - protein, 30 aa, synthetic construct
TRSSRAGLQFPVGRVHRLLRKRPRPDDLEI

SEQ ID NO: 61 - protein, 35 aa, synthetic construct
GWTLNSAGYLLGKINKALAALAKKILRPRPDDLEI

SEQ ID NO: 62 - protein, 27 aa, synthetic construct
KLALKLALKALKAALKLARPRPDDLEI

SEQ ID NO: 63 - protein, 25 aa, synthetic construct
AAVALLPAVLLALLAPRPRPDDLEI

SEQ ID NO: 64 - protein, 19 aa, synthetic construct
VPMLKPMLKERPRPDDLEI

SEQ ID NO: 65 - protein, 37 aa, synthetic construct
MANLGYWLLALFVTMWTDVGLCKKRPKPRPRPDDLEI

SEQ ID NO: 66 - protein, 27 aa, synthetic construct
LLIILRRRIRKQAHAHSKRPRPDDLEI

SEQ ID NO: 67 - protein, 30 aa, synthetic construct
KETWWETWWTEWSQPKKKRKVRPRPDDLEI

SEQ ID NO: 68 - protein, 27 aa, synthetic construct
RGGRLSYSRRRFSTSTGRRPRPDDLEI

SEQ ID NO: 69 - protein, 24 aa, synthetic construct
SDLWEMMMVSLACQYRPRPDDLEI

SEQ ID NO: 70 - protein, 21 aa, synthetic construct
TSPLNIHNGQKLRPRPDDLEI

SEQ ID NO: 71 - protein, 120 aa, synthetic construct
KGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVD

SEQ ID NO: 72 - protein, 120 aa, synthetic construct
KTDPYSHSGTMSPSKDCGSPKYAYYNGCSSPTAPLSPMSPPGYKLVTGDRNNSSCRNYNKQASEQNWANYSAEQNRMGQAGSTISNSHAQPFDFADEHQNTKKLASGHELQPLTIVDQRP

SEQ ID NO: 73 - protein, 120 aa, synthetic construct
LGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKV

SEQ ID NO: 74 - protein, 120 aa, synthetic construct
GFGTIRDTLNNKRKELEDSGTYNYPFTWNTPSAPPGYNIAVKPDQMQYTELSNAKMAYKQNKANIAQEQQYGSNEENIPADLENLQREIKVAQERLDMAIQAYNNQNNPGSSSREKKSKA

SEQ ID NO: 75 - protein, 120 aa, synthetic construct
PYLVDCFVSRPTEKTIFIIFMLVVGLISLVLNLLELVHLLCRCLSRGMRARQGQDAPPTQGTSSDPYTDQVFFYLPVGQGPSSPPCPTYNGLSSSEQNWANLTTEERLASSRPPLFLDPP

SEQ ID NO: 76 - protein, 120 aa, synthetic construct
CGSKEHGNRKMRGRLLLTYMASIFFKSVFEVAFLLIQWYLYGFTLSAVYICEQSPCPHRVDCFLSRPTEKTIFILFMLVVSMVSFVLNVIELFYVLFKAIKNHLGNEKEEVYCNPVELQK

SEQ ID NO: 77 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 78 - DNA, 60 bp, synthetic construct
  source 1..60: mol_type = other DNA
ccctcctcccgggcctcctcccgggcctcctcccggccccggcccgacgacctggagatc

SEQ ID NO: 79 - DNA, 27 bp, synthetic construct
  source 1..27: mol_type = other DNA
cggccccggcccgacgacctggagatc

SEQ ID NO: 80 - DNA, 27 bp, synthetic construct
  source 1..27: mol_type = other DNA
cggccccggcccgacgacctggaggtg

SEQ ID NO: 81 - DNA, 27 bp, synthetic construct
  source 1..27: mol_type = other DNA
cggccccggcccgacgacgtgcccgtg

SEQ ID NO: 82 - DNA, 27 bp, synthetic construct
  source 1..27: mol_type = other DNA
aaggcccggtccgacgacctgtccgtg

SEQ ID NO: 83 - DNA, 48 bp, synthetic construct
  source 1..48: mol_type = other DNA
cggcagcccaagatctggttccccaaccggcggaagccctggaagaag

SEQ ID NO: 84 - DNA, 108 bp, synthetic construct
  source 1..108: mol_type = other DNA
cggcagcccaagatctggttccccaaccggcggaagccctggaagaagccctcctcccgggcctcctcccgggcctcctcccggccccggcccgacgacctggagatc

SEQ ID NO: 85 - DNA, 75 bp, synthetic construct
  source 1..75: mol_type = other DNA
cggcagcccaagatctggttccccaaccggcggaagccctggaagaagcggccccggcccgacgacctggagatc

SEQ ID NO: 86 - DNA, 75 bp, synthetic construct
  source 1..75: mol_type = other DNA
cggcagcccaagatctggttccccaaccggcggaagccctggaagaagcggccccggcccgacgacctggaggtg

SEQ ID NO: 87 - DNA, 75 bp, synthetic construct
  source 1..75: mol_type = other DNA
cggcagcccaagatctggttccccaaccggcggaagccctggaagaagcggccccggcccgacgacgtgcccgtg

SEQ ID NO: 88 - DNA, 75 bp, synthetic construct
  source 1..75: mol_type = other DNA
cggcagcccaagatctggttccccaaccggcggaagccctggaagaagaaggcccggtccgacgacctgtccgtg

SEQ ID NO: 89 - protein, 20 aa, synthetic construct
PCSRASSRMSSRARPDDLDV

SEQ ID NO: 90 - protein, 19 aa, synthetic construct
PRVSVPNFGRTQSSDSAYV

SEQ ID NO: 91 - protein, 26 aa, synthetic construct
  SITE 1: note = biotinylated
  SITE 1: note = Nle
XRQPKIWFPNRRKPWKKRPRPDDLEI

(19) United States
(12) Patent Application Publication
(10) Pub. No.: US 2025/0281574 A1
(43) Pub. Date: Sep. 11, 2025

(54) COMPOSITIONS AND METHODS FOR TREATING COMPLICATIONS OF VIRAL INFECTIONS AND OTHER RESPIRATORY DISORDERS

(71) Applicant: Xequel Bio, Inc., Fort Worth, TX (US)

(72) Inventors: Carissa JAMES, Mt. Pleasant, SC (US); Christina L. GREK, Mt. Pleasant, SC (US)

(21) Appl. No.: 18/861,250

(22) PCT Filed: Feb. 28, 2023
(86) PCT No.: PCT/US2023/063409

§ 371 Date: Oct. 29, 2024

Related U.S. Application Data
(60) Provisional application No. 63/336,872, filed on Apr. 29, 2022.

(51) Int. Cl. A61K 38/17; A61K 9/00; A61K 31/706; A61P 11/00; A61P 31/14; C07K 14/705

(52) U.S. Cl. CPC A61K 38/177; A61K 9/0078; A61K 31/706; A61P 11/00; A61P 31/14; C07K 14/705; C07K 2319/01

(57) ABSTRACT

In one aspect, the present disclosure relates to treating or preventing a respiratory disease or disorder, by administering one or more compositions comprising an isolated polypeptide derived from an alpha Connexin and an anti-viral agent such as, for example, nucleoside analogs. Exemplary respiratory diseases or disorders include acute respiratory distress syndrome (ARDS), alcoholic lung syndrome, acute lung injury (ALI), pulmonary fibrosis, idiopathic pulmonary fibrosis (IPF), and/or chronic obstructive pulmonary disease (COPD). In some aspects, the respiratory disease or disorder is a complication of a respiratory viral disease.

### CROSS-REFERENCE TO RELATED APPLICATIONS

This application claims priority from U.S. Provisional Application No. 63/336,872, filed on Apr. 29, 2022, which is incorporated by reference herein in its entirety for all purposes.

### STATEMENT REGARDING FEDERAL FUNDING

This invention was made with government support under contract W81XWH2110158 awarded by the Department of Defense. The government has certain rights in the invention.

### DESCRIPTION OF THE TEXT FILE SUBMITTED ELECTRONICALLY

The Sequence Listing XML associated with this application is provided in XML file format and is hereby incorporated by reference into the specification. The name of the XML file containing the Sequence Listing XML is FIRS_014_01WO_SeqList_ST26.xml. The XML file is 82,854 bytes, was created on Feb. 25, 2023, and is being submitted electronically via U.S. Patent Center.

### BACKGROUND OF THE INVENTION

Most patients suffering from respiratory viral pneumonia will recover. However, a significant number of those affected, especially older patients and those with chronic underlying conditions, are at high risk of developing severe pneumonia and life-threatening acute respiratory distress syndrome (ARDS), a severe form of pulmonary edema that precedes respiratory failure and multiple organ dysfunction. ARDS patients require resource intensive critical care including mechanical ventilation and extended management in intensive care units. Mortality rate in ARDS patients remains ˜40% despite the availability of state-of-the-art intensive care medicine. Compounding the dismal mortality associated with ARDS, there is likely to be a significant shortage of ventilators and health care workers trained to use them in the United States during a large-scale pandemic, such as what occurred with Coronavirus Disease (COVID-19) in 2019-2020.

There is a clear need in the art for an effective treatment for infection-induced lung injury that reduces ARDS incidence, need for mechanical ventilation, and deaths.

### BRIEF SUMMARY OF THE INVENTION

In one aspect, the present disclosure provides methods for treating or preventing a respiratory disease or disorder, comprising administering to the subject a composition comprising an isolated polypeptide derived from an alpha Connexin in combination with administering an anti-viral agent. In some cases, the anti-viral agent is present in a composition. In some cases, the anti-viral agent is present in the same composition as the isolated polypeptide derived from an alpha Connexin. In some cases, the anti-viral agent is present in a different composition than the isolated polypeptide derived from an alpha Connexin. In some cases, the composition comprising the isolated polypeptide derived from an alpha Connexin is administered at the same time or simultaneously with the administration of the anti-viral agent. In some cases, the composition comprising the isolated polypeptide derived from an alpha Connexin is administered prior to or after the administration of the anti-viral agent. In some cases, the anti-viral agent prevents viral replication. In some cases, the anti-viral agent is an inhibitor of a viral RNA-dependent, RNA polymerase. In some cases, the anti-viral agent is a prodrug or a protide. In some cases, the anti-viral agent is a nucleoside analog. In some cases, the nucleoside analog is an adenosine analog. In some cases, the nucleoside analog is remdesivir. In some cases, the nucleoside analog is an active metabolite thereof. In some cases, the nucleoside analog is a derivative of remdesivir. In some cases, the derivative of remdesivir is selected from the group consisting of ATV006, GS-621763 and VV116. In some embodiments, the respiratory disease or disorder is associated with inflammation and/or fibrosis of the lung. In some embodiments, the respiratory disease or disorder is pneumonia, acute respiratory distress syndrome (ARDS), alcoholic lung syndrome, acute lung injury (ALI), pulmonary fibrosis, idiopathic pulmonary fibrosis (IPF), and/or chronic obstructive pulmonary disease (COPD).

In some embodiments, the present disclosure provides compositions and methods for treating or preventing a complication of a respiratory viral disease in a subject, comprising administering to the subject a composition comprising an isolated polypeptide derived from an alpha Connexin and administering an anti-viral agent. In some cases, the anti-viral agent is present in a composition. In some cases, the anti-viral agent is present in the same composition as the isolated polypeptide derived from an alpha Connexin. In some cases, the anti-viral agent is present in a different composition than the isolated polypeptide derived from an alpha Connexin. In some cases, the composition comprising the isolated polypeptide derived from an alpha Connexin is administered at the same time or simultaneously with the administration of the anti-viral agent. In some cases, the composition comprising the isolated polypeptide derived from an alpha Connexin is administered prior to or after the administration of the anti-viral agent. In some cases, the anti-viral agent prevents viral replication. In some cases, the anti-viral agent is an inhibitor of a viral RNA-dependent, RNA polymerase. In some cases, the anti-viral agent is a prodrug or a protide. In some cases, the anti-viral agent is a nucleoside analog. In some cases, the nucleoside analog is an adenosine analog. In some cases, the nucleoside analog is remdesivir. In some cases, the nucleoside analog is an active metabolite thereof. In some cases, the nucleoside analog is a derivative of remdesivir. In some cases, the derivative of remdesivir is selected from the group consisting of ATV006, GS-621763 and VV116. Further, provided herein are compositions for use in treating or preventing a complication of a respiratory viral disease in a subject, wherein the composition comprises an isolated peptide derived from an alpha Connexin and an anti-viral agent. In some cases, the anti-viral agent prevents viral replication. In some cases, the anti-viral agent is an inhibitor of a viral RNA-dependent, RNA polymerase. In some cases, the anti-viral agent is a prodrug or a protide. In some cases, the anti-viral agent is a nucleoside analog. In some cases, the nucleoside analog is an adenosine analog. In some cases, the nucleoside analog is remdesivir. In some cases, the nucleoside analog is an active metabolite thereof. In some cases, the nucleoside analog is a derivative of remdesivir. In some cases, the derivative of remdesivir is selected from the group consisting of ATV006, GS-621763 and VV116. In some embodiments, the respiratory viral disease is caused by severe acute respiratory syndrome-Coronavirus 2 (SARS-CoV-2). In some embodiments, the complication of the respiratory viral disease is lung inflammation, pulmonary edema, alveolar hemorrhage, ARDS and/or acute lung injury (ALI). For example, in some embodiments, the compositions and methods provided herein are for treatment or prevention of virus (e.g., SARS-CoV-2, influenza)-induced ARDS and/or virus (e.g., SARS-CoV-2, influenza)-induced ALI and/or virus (e.g., SARS-CoV-2, influenza)-induced pulmonary fibrosis.

In some embodiments, the polypeptide comprises the carboxy terminal-most 4 to 30 contiguous amino acids of the alpha Connexin. In some embodiments, the alpha Connexin is Connexin 37, Connexin 40, Connexin 43, or Connexin 45. In some embodiments, the polypeptide comprises an amino acid sequence selected from the group consisting of SEQ ID NO: 1, SEQ ID NO: 2, SEQ ID NO: 3, SEQ ID NO: 4, and SEQ ID NO: 5. In one embodiment, the polypeptide comprises the amino sequence of SEQ ID NO: 2. In some embodiments, the polypeptide further comprises a cellular internalization sequence. In some embodiments, the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol). In some embodiments, the cellular internalization sequence is Antennapedia, and wherein the sequence comprises the amino acid sequence of SEQ ID NO:7. In some embodiments, the polypeptide comprises an amino acid sequence selected from the group consisting of SEQ ID NO:8, SEQ ID NO:9, SEQ ID NO: 10, SEQ ID NO:11, and SEQ ID NO: 12. In one embodiment, the polypeptide comprises the amino acid sequence of SEQ ID NO: 9.

In some embodiments, polypeptide and/or the anti-viral agent is/are administered to the subject orally, parenterally, intranasally, intratracheally, by inhalant, or by topical intranasal administration. In some embodiments, the polypeptide and/or the anti-viral agent is/are administered to the subject via aerosolized delivery. In some embodiments, the polypeptide and/or the anti-viral agent is/are administered via an inhaler device. In some embodiments, the polypeptide and/or the anti-viral agent is/are administered via a dry powder inhaler, a metered dose inhaler, or a nebulizer. In some embodiments, the polypeptide and/or the anti-viral agent is/are administered via a ventilator. In some embodiments, the polypeptide and/or the anti-viral agent is/are administered to the subject in a drug loaded microcarrier formulation, such as nanoparticles or exosomes. In some cases, the anti-viral agent is administered intravenously. In some cases, the anti-viral agent is administered orally. In some cases, the anti-viral agent prevents viral replication. In some cases, the anti-viral agent is an inhibitor of a viral RNA-dependent, RNA polymerase. In some cases, the anti-viral agent is a prodrug or a protide. In some cases, the anti-viral agent is a nucleoside analog. In some cases, the nucleoside analog is an adenosine analog. In some cases, the nucleoside analog is remdesivir. In some cases, the nucleoside analog is an active metabolite thereof. In some cases, the nucleoside analog is a derivative of remdesivir. In some cases, the derivative of remdesivir is selected from the group consisting of ATV006, GS-621763 and VV116.

In some embodiments, the polypeptide and/or the anti-viral agent is/are administered to the subject at the onset of infection. In some embodiments, the polypeptide and/or the antiviral agent is/are administered to the subject after infection with a virus that causes the respiratory viral disease and prior to the onset of symptoms of the respiratory viral disease. Accordingly, in some embodiments, the polypeptide and/or the anti-viral agent is/are administered after the subject is identified as having been infected with the virus (e.g., SARS-CoV-2) or after the subject has been identified as being suspected of being infected with the virus, or as being at risk of infection with the virus. In some embodiments, the polypeptide and/or the anti-viral agent is/are administered prior to infection with the virus. In some embodiments, the polypeptide and/or the anti-viral agent is/are administered prior to the onset of symptoms of the respiratory viral disease. In some embodiments, the polypeptide and/or the anti-viral agent is/are administered after the onset of symptoms of the respiratory viral disease. In some embodiments, the compositions and methods provided herein prevent the onset of, or mitigate the progression of, or reverse progression of, the respiratory viral disease. In some embodiments, the compositions and methods provided herein maintain lung function after the onset of the respiratory viral disease. In some embodiments, the compositions and methods provided herein ameliorate or reduce lung inflammation, alveolar hemorrhage, and/or pulmonary edema as compared to treatment with either the polypeptide or anti-viral agent alone. In some cases, the anti-viral agent prevents viral replication. In some cases, the anti-viral agent is an inhibitor of a viral RNA-dependent, RNA polymerase. In some cases, the anti-viral agent is a prodrug or a protide. In some cases, the anti-viral agent is a nucleoside analog. In some cases, the nucleoside analog is an adenosine analog. In some cases, the nucleoside analog is remdesivir. In some cases, the nucleoside analog is an active metabolite thereof. In some cases, the nucleoside analog is a derivative of remdesivir. In some cases, the derivative of remdesivir is selected from the group consisting of ATV006, GS-621763 and VV116.

In some embodiments, the compositions and methods provided herein treat or prevent lung injury and/or respiratory disorders. Exemplary indications in acute and chronic lung injuries and/or respiratory disorders include pneumonia, ARDS, alcoholic lung injury, ALI, pulmonary fibrosis, idiopathic pulmonary fibrosis (IPF), and chronic obstructive pulmonary disease (COPD). Thus, in some embodiments, the compositions and methods provided herein treat or prevent ARDS, alcoholic lung syndrome, and/or ALI. In some embodiments, the compositions and methods provided herein treat or prevent SARS-CoV-2 related pneumonia, ARDS, ALI, and/or pulmonary fibrosis.

### BRIEF DESCRIPTION OF THE DRAWINGS

FIG. 1 provides a schematic view of the aCT1 peptide.

FIG. 2A. shows that intranasal pre- and post-treatment with aCT1 provides significant protection against lipopolysaccharide (LPS)-induced mortality. C57BL/6 mice were pretreated with either 5.76 mg/kg aCT1 peptide, 5.76 mg/kg control peptide, or PBS vehicle control prior to inoculation with a lethal dose (35 mg/kg) of LPS. A further cohort was treated six (6) hours post inoculation of LPS, as shown in FIG. 2B. For FIG. 2A, n=20-22/group and p<0.0001 by Log-rank Mantel-Cox test; for FIG. 2B, n=5-8/group and p<0.001 by Log-rank Mantel-Cox test. Doses are reported as presented dose, with estimated animal body weight of 25 grams.

FIGS. 3A-3B shows that aCT1 prolongs survival in a mouse model of sepsis. Mice were administered vehicle control, 14.34 mg/kg control peptide, or 14.34 mg/kg of aCT1 peptides immediately after cecal-ligation and puncture (CLP) procedure, as shown in FIG. 3A. A further cohort was treated six (6) hours post CLP procedure, as shown in FIG. 3B. For FIG. 3A, n=7-9/group and p=0.052 by Log-rank Mantel-Cox test; for FIG. 3B, n=8/group and p<0.05 by Log-rank Mantel-Cox test. Doses are reported as administered dose, with estimated animal body weight of 25 grams.

FIG. 4 shows that aCT1 reduces intra-alveolar neutrophil accumulation. Histopathological analysis using hematoxylin and eosin (H&E) staining of lungs at 12 hours post CLP injury shows delivery of 14.34 mg/kg aCT1 significantly decreased immune cell infiltration and alveolar edema. Representative images are shown.

FIGS. 5A-5B shows that normal lung function is not impaired by aerosolized aCT1 treatment in healthy animals. Delivery of 2.88 mg/kg aCT1 was not associated with alterations in lung function or overall respiratory health in healthy mice, as measured by enhanced pause (Penh). Treatment with aCT1 did not result in altered Penh three (3) hours after nebulization (FIG. 5A) or six (6) hours after nebulization (FIG. 5B).

FIG. 6A provides immunostaining images demonstrating that aCT1 localized to epithelial cells and endothelial cells in the conducting airways and alveoli. FIG. 6B shows quantification of stained Cx43 phosphorylated at S368 in bronchial and alveolar epithelial cells, as an indication of aCT1 activity. (n=5; #p<0.0001).

FIG. 7 shows that pre-treatment of normal human bronchial epithelial (NHBE) cells with aCT1 prevents oxidative stress induced pulmonary injury. Pre-treatment significantly increased TER readings after exposure to 500 mM H₂0₂, indicative of improved barrier function. (n=3/group; p-values reported at 72 hours compared to H₂O₂).

FIG. 8 shows that aCT1 provides protection from LPS induced injury. Normal human bronchial epithelial (NHBE) cells were exposed to 10 μg LPS. Cells pre-treated with aCT1 significantly improved TER readings, indicative of improved barrier function. (n=3/group; p-values reported at 72 hours compared to LPS).

FIGS. 9A-9C illustrate the results of aCT1 in combination with remdesivir in a Syrian hamster model of SARS-CoV-2 infection. FIG. 9A shows that aerosolized aCT1 in combination with remdesivir mitigates inflammatory cell infiltration into the lung following SARS-CoV-2 infection. Targeted delivery of 10 μg/kg (low) or 20 μg/kg (high) aCT1 via nebulizer in combination with 15 mg/kg remdesivir resulted in decreased immune cell infiltration into lung tissue following SARS-CoV-2 challenge of Syrian hamsters. FIG. 9B shows that aerosolized aCT1 in combination with remdesivir prevented an increase in lung:body weight ratio, which is indicative of pulmonary edema. FIG. 9C shows that aerosolized aCT1 in combination with remdesivir results in less alveolar hemorrhage. (n=4-8/group; *p<0.05; **p<0.01; ***p<0.001; ****p<0.0001 compared to Challenge group).

FIG. 10 shows histopathology of hematoxylin and eosin (H&E) stained lung tissue from the experimental and control groups described in Example 6 and FIGS. 9A-9B. The lung tissue slices show representative effects on inflammation and edema from the SARS-CoV-2 virus infection. Lung tissue treated with remdesivir and in combination with aCT1 exhibit decreased alveolar edema and inflammation.

FIG. 11 illustrates the effects of aerosolized aCT1 peptide in a mouse model of alcoholic lung syndrome. FIG. 11 shows that aerosolized aCT1 prevented alcohol-induced permeability in the lung. C57BL/6 mice were fed ethanol at increasing concentrations, then maintained on 20% ethanol in drinking water for an additional 10 weeks. Mice were treated with 5 mg/kg aerosolized aCT1 peptide one (1) hour after intratracheal instillation of 5 mg/kg LPS to induce lung injury. Evan's Blue assay indicated that aCT1 treatment reduced pulmonary barrier permeability back to non-alcoholic levels. (n=9-16/group; *p<0.05; **p<0.01).

FIG. 12 shows in vitro normal human bronchial epithelial cells (NHBEs) treated with 100 mM alcohol. When treated with aCT1 peptide, cells demonstrated improved electrical resistance, indicative of improved barrier function. (n=3/group; **p<0.01).

### DETAILED DESCRIPTION

As used herein, the term “a” or “an” can refer to one or more of that entity, i.e. can refer to a plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

The term “remdesivir” as used herein can be denoted in a broad sense to include not only “remdesivir” per se but also its pharmaceutically acceptable derivatives thereof. Suitable pharmaceutically acceptable derivatives can include pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

Provided herein are methods for treating or preventing respiratory diseases and disorders such as, for example, those caused by viral respiratory infections. Provided herein are methods for treating or preventing diseases and disorders that are complications of viral respiratory infections. In some cases, the respiratory disease or disorder is caused by a viral infection. The viral respiratory infection can be caused by any virus known to cause respiratory infections. In some cases, the virus known to cause respiratory infections is an RNA virus or a DNA virus. In some embodiments, the respiratory viral infection is caused by a virus selected from Severe Acute Respiratory Syndrome-Corona Virus (SARS-COV), Middle East Respiratory Syndrome virus (MERS-COV), human HCoV-229E, HCoV-OC43, HCoV-NL63 and HCoV-HKU1. In some embodiments, the virus infection is a coronavirus infection. In some embodiments, the coronavirus is an alpha coronavirus (e.g., HCoV-EE29, HCoV-NL63) or a beta coronavirus (e.g., HCoV-0C43, HCoV-HKU1, MERS-COV, or SARS-COV). In some embodiments, the coronavirus infection is SARS-COV (e.g., SARS-CoV-1, SARS-CoV-2). In one embodiment, the SARS-COV is SARS-CoV-2 (virus causing COVID-19). In some embodiments, the respiratory viral infection is an influenza viral infection or a parainfluenza virus infection (PIV) infection. In some embodiments, the influenza viral infection is selected from the group consisting of Influenza A, Influenza B, and Influenza C viral infections. In some embodiments, the Influenza A virus comprises H1N1, H2N2, H3N2, H5N1, H7N7, H1N2, H9N2, H7N2, H7N3, or H10N7 subtypes. In some embodiments, the respiratory viral infection is respiratory syncytial virus (RSV).

The highly pathogenic SARS-CoV-2 is associated with rapid virus replication, massive inflammatory cell infiltration and elevated pro-inflammatory cytokine/chemokine responses. Infection and the ensuing inflammatory response can result in acute lung injury (ALI) and lead to acute respiratory distress syndrome (ARDS), pulmonary fibrosis, and death. FDA has approved the antivirals remdesivir, molnupiravir, and nirmatrelvir/ritonavir as well as immune modulators baricitinib and tocilizumab to treat specified patient populations with COVID-191. Once COVID-19 progresses to severe disease, patients undergo supportive care including mechanical ventilation, which can itself further exacerbate respiratory distress. Mortality rates in patients requiring mechanical ventilation are high.

Interconnected epithelial cells line the pulmonary air spaces, forming a physiological barrier separating inspired air from fluid-filled tissues and providing a surface for gas exchange. Integrity of this barrier is essential for pulmonary function; where disruption results in the accumulation of fluid in the alveoli and respiratory failure². SARS-CoV-2, similar to the previous coronaviruses MERS-COV and SARS-COV, targets epithelial cells lining the airways for viral entry and replication^{3, 4}. The virus causes severe lesions and shedding of the bronchial and alveolar epithelial cells lining airways⁵. The resulting diffuse alveolar damage primes the lung for edema and fibrosis. In many patients infected with SARS-CoV-2, particularly those in high risk groups, this progresses to a severe pulmonary pneumonia and acute respiratory distress as the lung becomes fluid filled and fibrotic⁶. Excessive inflammation elicited in response to viral infection and lung injury exacerbates the severity of COVID-19 as proinflammatory cytokines and immune cell infiltration exacerbate lung fibrosis and thickening of the airway walls, further compromising the lung's ability to permit gas exchange⁷.

Severe pneumonia, resulting from loss of pulmonary epithelial barrier function and a faulty immune response, results in disease progression to acute respiratory distress syndrome (ARDS) in many patients with respiratory viral infections, such as those caused by SARS Coronaviruses or influenza. Without wishing to be bound by theory, the alpha Connexin peptides provided herein provide a therapeutic intervention that preserves epithelial integrity and dampens inflammation in infected lungs. Accordingly, the methods provided herein include mitigation of development of acute respiratory failure in hospitalized patients (e.g., patients having respiratory viral infections, such as those caused by SARS coronaviruses or influenza), which lessens the need for intensive mechanical ventilation and dramatically improves patient survival.

Without wishing to be bound by theory, alpha Connexin peptides provided herein directly target and repair the damaged cells lining airspaces and vasculature in the virus infected lung, thus restoring barrier integrity and directly addressing the cause of ARDS. While antiviral therapies in development for respiratory viral infections can limit viral replication and accelerate viral clearance, these therapies will not address the need for rapid repair of the pulmonary epithelium in critically ill patients to prevent progression of severe pneumonia to ARDS. In some embodiments, therapeutically targeting intercellular junctions in the infected lung via the alpha Connexin peptide (e.g., aCT1) stabilizes epithelial barriers, mitigates the pathological immune response, and prevents development of acute respiratory failure to improve survival and decrease need for ventilation.

Accordingly, in some embodiments, the present disclosure provides methods for or compositions for use in treating a respiratory disease, disorder, or condition by administering to a subject in need thereof a polypeptide provided herein (e.g., an alpha Connexin polypeptide, e.g., aCT polypeptide) in combination with administering an anti-viral agent. In some cases, the anti-viral agent is present in a composition. In some cases, the anti-viral agent is present in the same composition as the polypeptide provided herein (e.g., an alpha Connexin polypeptide, e.g., aCT polypeptide). In some cases, the anti-viral agent is present in a different composition than the polypeptide provided herein (e.g., an alpha Connexin polypeptide, e.g., aCT polypeptide). In some cases, the polypeptide provided herein (e.g., an alpha Connexin polypeptide, e.g., aCT polypeptide) is administered at the same time or simultaneously with the administration of the anti-viral agent. In some cases, the polypeptide provided herein (e.g., an alpha Connexin polypeptide, e.g., aCT polypeptide) is administered prior to or after the administration of the anti-viral agent. In some cases, the anti-viral agent is a prodrug. In some cases, the anti-viral agent is any agent that inhibits viral replication. In some cases, the anti-viral agent is a member of a class of compounds that inhibits or alters the function of a viral RNA-dependent, RNA polymerase (RdRp). An inhibitor of RdRp for use in a method or composition provided herein can be any such compound described in WO/2021/155119, which is herein incorporated by reference. In some cases, the anti-viral agent is a member of the nucleoside analog class of chemical compounds. The nucleoside analogs can be purine nucleoside analogs or pyrimidine nucleoside analogs. In some cases, the anti-viral agent is a member of the nucleotide analog class of chemical compounds. In some cases, the anti-viral agent is an active metabolite of a prodrug. In some cases, the anti-viral agent is a protide. In some cases, the anti-viral agent is a derivative of a known nucleotide or nucleoside analog. The derivative can be an ester prodrug. In some cases, the anti-viral agent is an adenosine analog, an active metabolite of an adenosine analog, or a derivative of an adenosine analog. In some cases, the anti-viral agent is an active metabolite of an adenosine analog. In one embodiment, the antiviral agent is remdesivir (GS-5734). In some cases, the antiviral agent is remdesivir, which can also be referred to as the tradename Veklury. In one embodiment, the antiviral agent is a metabolite of remdesivir. The metabolite can be GS-441524. The metabolite can be the active adenosine nucleoside triphosphate metabolite GS-443902. The metabolite of remdesivir can be any metabolite described in WO/2021/231361A1, which is herein incorporated by reference. In one embodiment, the antiviral agent is a derivative of remdesivir. The derivative of remdesivir can be an ester prodrug. In some case, the derivative of remdesivir is selected from the group consisting of ATV006, GS-621763 and VV116. In one embodiment, the antiviral agent is an isoform of remdesivir. The isoform of remdesivir can be any of the isoforms described in US20210228605, which is herein incorporated by reference. In one embodiment, the antiviral agent is an analog of remdesivir. The analog of remdesivir can be any of the isoforms described in WO/2021/202907, which is herein incorporated by reference. In one embodiment, the antiviral agent is a crystalline of remdesivir. The analog of remdesivir can be any of the isoforms described in WO/2021/248229A1, which is herein incorporated by reference. In one embodiment, the antiviral agent is a salt of remdesivir. The analog of remdesivir can be any of the isoforms described in WO/2022/020940A1, which is herein incorporated by reference. In some cases, the anti-viral agent is a guanosine analog, an active metabolite of a guanosine analog, or a derivative of a guanosine analog. In some cases, the anti-viral agent is a cytidine analog, an active metabolite of a cytidine analog or a derivative of a cytidine analog. The cytidine analog can be molnupiravir. The cytidine analog can be an active metabolite of molnupiravir. The cytidine analog can be a derivative of molnupiravir. In some cases, the anti-viral agent can be a uridine analog, an active metabolite of a uridine analog or a derivative of a uridine analog. In some cases, the anti-viral agent can be a thymidine analog, an active metabolite of a thymidine analog, or a derivative of a thymidine analog. In some cases, an anti-viral agent that is nucleoside/nucleotide analog in nature that can be used in the methods or compositions provided herein can be selected from the group consisting of remdesivir, molnupiravir, ATV-006, GS-621763, VV116, GS-443902, GS-441524, 2′-C-methylguanosine, 2′-C-methyladenosine, 7-Deaza-2′-C-methyl-adenosine, INX-08189, 2′-C-methylcytidine, sofosbuvir, 2′-C-methyluridine, 2′-C-ethynyladenosine, NITD008, NITD449, NITD203, 4′-C-azidocytidine, balapiravir, ganciclovir, valganciclovir, brivudine, entecavir, RO-9187, BCX4430, T-1106, 6-Methyl-7-deazaadenosine, N6-(9-antranylmethyl) adenosine, N6-(1-pyrenylmethyl) adenosine, N6-benzyl-5′-O-triisopropylsilyl adenosine, N6-benzyl-5′-O-trityl adenosine, N6-benzyl-5′-O-tert-butyldimethylsilyl-adenosine, 2′,5′Di-O-trityluridine, 3′,5′Di-O-trityluridine, ribavirin, favipiravir, GRL-002, GRL-003, Flex 1, Flex 2, ribavirin, valacyclovir, famciclovir, acyclovir, penciclovir, vidarabine, telbivudine, cidofovir, adefovir dipivoxil, zidovudine, azidothymidine, didanosine, zalcitabine, stavudine, lamivudine, abacavir, emtricitabine, tenofovir disoproxil fumarate, idoxuridine, trifluridine, ETAR, IM18, 6-Azauridine, 5-(Perylen-3-yl) ethynyl-arabino-uridine, 5-(Perylen-3-yl) ethynyl-2′-deoxy-uridine, 5-(Pyren-1-yl) ethynyl-2′-deoxy-uridine and any combinations thereof.

In some embodiments, the respiratory disease, disorder or condition is COVID-19 (coronavirus disease) mediated acute respiratory distress syndrome (ARDS). In some embodiments, the respiratory disease, disorder, or condition is ARDS which is not associated with a viral infection, or which is not triggered by a viral infection. In some embodiments, the respiratory disease, disorder, or condition is ARDS that is triggered by a secondary insult that may or may not be a viral infection. For example, In some embodiments, the respiratory disease, disorder, or condition may be an underlying condition or may be associated with an underlying condition, wherein a secondary insult (e.g., a lung injury, a traumatic brain injury and/or viral respiratory infection) may result in ARDS. In some embodiments, the respiratory disease or condition is alcoholic lung injury or alcoholic lung syndrome. In some embodiments, the respiratory disease or condition is an acute lung injury. The acute lung injury can be induced by a traumatic brain injury. In some embodiments, the respiratory disease or condition is pulmonary fibrosis, IPF, or COPD. The compositions and methods provided herein are useful for treatment of chronic lung injuries and/or respiratory disorders (e.g., pneumonia, pulmonary fibrosis, IPF, COPD, ARDS, and/or ALI) whether or not associated with or triggered by a viral infection.

In some embodiments, the respiratory disease, disorder or condition is lung inflammation, alveolar hemorrhage or pulmonary edema. In some embodiments, the lung inflammation, alveolar hemorrhage or pulmonary edema is triggered by a viral infection. The viral infection can be caused by any virus known in the art and/or provided herein to cause a viral respiratory infection. Further to these embodiments, provided herein are methods for or compositions for use in treating or preventing alveolar hemorrhage, lung inflammation or pulmonary edema that comprises administering to a subject suffering from or suspected of suffering from alveolar hemorrhage, lung inflammation or pulmonary edema a composition comprising an isolated polypeptide comprising an alpha connexin and a composition comprising an anti-viral agent. The alpha connexin can be any alpha connexin peptide provided herein such as, for example, the carboxy terminal-most 4 to 30 contiguous amino acids of an alpha Connexin. The anti-viral agent can be any anti-viral agent provided herein, such as, for example, a nucleoside analog (e.g., remdesivir or an active metabolite or derivative of remdesivir). The alpha connexin and the anti-viral agent can be in the same or each in different compositions as provided herein. The administration of the combination of alpha connexin and anti-viral agent can act synergistically to reduce or ameliorate the alveolar hemorrhage, the lung inflammation and/or the pulmonary edema more than administration of either the alpha connexin or the anti-viral agent alone. In some cases, the anti-viral agent may not or may not be known for producing any direct effect on alveolar hemorrhage, lung inflammation or pulmonary edema. Administration of the combination may reduce or ameliorate alveolar hemorrhage, lung inflammation and/or pulmonary edema by at least, at most or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 125%, 150%, 175% or 200% as compared to administration of either the alpha connexin or the anti-viral agent alone. Administration of the combination may reduce or ameliorate alveolar hemorrhage, lung inflammation and/or pulmonary edema by between 1%-5%, 5%-10%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-100%, 100%-125%, 125%-150%, 150%-175% or 175%-200% as compared to administration of either the alpha connexin or the anti-viral agent alone.

In some embodiments, the present disclosure provides compositions and methods for treating or preventing a respiratory disease, disorder, or condition, comprising administering a composition provided herein directly into the lungs via nebulization, a combination of nebulization and oral administration, or a combination of nebulization and intravenous administration. In some embodiments, the present disclosure provides compositions and methods for treating or preventing a respiratory disease, disorder, or condition, comprising administering a composition provided herein into the lungs via inhalation, a combination of inhalation and oral administration, or a combination of inhalation and intravenous administration.

Impaired wound healing and a dysfunctional immune response render the alcoholic lung unable to repair its chronically damaged epithelium. The cumulative effect of chronic alcohol ingestion on pulmonary gap and tight junctions causes a loss of barrier integrity that defines alcoholic lung syndrome, priming the lung for acute lung injury and ARDS. Alcoholic lung syndrome often remains undiagnosed until hospitalization due to a secondary insult, as compensatory upregulation of fluid transport in the alcoholic lung allows the syndrome to remain subclinical. When faced with an acute secondary insult, these compensatory mechanisms are quickly overwhelmed, and the alcohol injured lung develops an exaggerated and lethal response to insult that precipitates respiratory failure. 25% of patients have alcohol use disorder at the time of hospital admission in the United States. In-hospital mortality for alcoholics is double that of matched non-alcoholic patients, and this is primarily attributed to the increased risk of developing ARDS. In some embodiments, the present disclosure provides methods for treating and/or preventing development of ARDS comprising administration of a composition comprising a polypeptide provided herein (e.g., aCT1) alone or in combination with an anti-viral agent as provided herein (e.g., remdesivir) to a patient upon hospital or ICU admission. In some embodiments, the patient is an alcoholic patient. Without wishing to be bound by theory, in some embodiments, compositions comprising a polypeptide provided herein (e.g., aCT1) alone or in combination with an anti-viral agent as provided herein (e.g., remdesivir) restore the pulmonary barrier in a patient having alcoholic lung injury and protect the lung from development of ARDS.

The polypeptides provided herein comprise a carboxy-terminal amino acid sequence of an alpha Connexin, or a conservative variant thereof. In some embodiments, the polypeptide comprises or consists of the amino acid sequence RPRPDDLEI (SEQ ID NO: 2). In some embodiments, the polypeptide is aCT1, as described herein. The term “aCT1” is used interchangeably herein with “aCT1” and “ACT1”. aCT1 is a 25 aa peptide having the amino acid sequence RQPKIWFPNRRKPWKKRPRPDDLEI (SEQ ID NO: 9). In some embodiments, the compositions and methods provided herein are related to preventing, treating, and/or mitigating the progression of complications from viral infections. In some embodiments, the compositions and methods provided herein are related to preventing, treating, and/or mitigating the progression of respiratory and pulmonary complications of viral infections, such as pneumonia, acute respiratory distress syndrome (ARDS) or acute lung injury (ALI), by administering aCT1 to a subject in need thereof. For example, in some embodiments, the compositions and methods herein are related to preventing, treating, and/or mitigating the progression of ARDS and/or ALI in patients suffering from a respiratory infection such as a SARS-CoV-2 infection, by administering aCT1 in combination with an anti-viral agent (e.g., a nucleoside analog such as remdesivir or active metabolites thereof or active derivatives thereof) to a subject in need thereof. In some embodiments, the aCT1 polypeptide provided herein used in combination with an anti-viral agent (e.g., a nucleoside analog such as remdesivir or active metabolites thereof or active derivatives thereof) is for use in preventing, treating, and/or mitigating the progression of respiratory and pulmonary complications of viral infections, such as pneumonia, ARDS or ALI. In some embodiments, provided herein are uses of aCT1 and an anti-viral agent (e.g., a nucleoside analog such as remdesivir or active metabolites thereof or active derivatives thereof) in the manufacture of a medicament for preventing or treating respiratory and pulmonary complications of viral infections, such as pneumonia, ARDS, ALI, and/or fibrosis.

The herein provided polypeptide can be any polypeptide comprising the carboxy-terminal most amino acids of an alpha Connexin, wherein the polypeptide does not comprise the full-length alpha Connexin protein. Thus, in some embodiments, the provided polypeptide does not comprise the cytoplasmic N-terminal domain of the alpha Connexin. In some embodiments, the provided polypeptide does not comprise the two extracellular domains of the alpha Connexin. In some embodiments, the provided polypeptide does not comprise the four transmembrane domains of the alpha Connexin. In some embodiments, the provided polypeptide does not comprise the cytoplasmic loop domain of the alpha Connexin. In some embodiments, the provided polypeptide does not comprise that part of the sequence of the cytoplasmic carboxyl terminal domain of the alpha Connexin proximal to the fourth transmembrane domain. There is a conserved proline or glycine residue in alpha Connexins consistently positioned some 17 to 30 amino acids from the carboxyl terminal-most amino acid. For example, for human Cx43 a proline residue at amino acid 363 is positioned 19 amino acids back from the carboxyl terminal-most isoleucine. In another example, for chick Cx43 a proline residue at amino acid 362 is positioned 18 amino acids back from the carboxyl terminal-most isoleucine. In another example, for human Cx45 a glycine residue at amino acid 377 is positioned 19 amino acids back from the carboxyl terminal most isoleucine. In another example for rat Cx33, a proline residue at amino acid 258 is positioned 28 amino acids back from the carboxyl terminal-most methionine. Thus, in some embodiments, the provided polypeptide does not comprise amino acids proximal to said conserved proline or glycine residue of the alpha Connexin. Thus, the provided polypeptide can comprise the c-terminal-most 4 to 30 amino acids of the alpha Connexin, including the c-terminal most 4, 5, 6, 7, 8, 9, 10, 11, 12, 13, 14, 15, 16, 17, 18, 19, 20, 21, 22, 23, 24, 25, 26, 27, 28, 29, 30 amino acids of the alpha Connexin. Exemplary alpha Connexin polypeptides are disclosed in U.S. Pat. Nos. 7,786,074; 7,888,319; 8,357,668; 8,809,257; 8,916,515; 8,859,733; 8,846,605; 9,161,984; 9,394,351; 9,408,381; 9,844,214; 9,855,313; 10,398,140; and 10,398,757, and/or International Patent Application No. PCT/US2018/000035, the entire contents of each of which are hereby incorporated by reference.

Connexins are the sub-unit protein of the gap junction channel, which is responsible for intercellular communication⁸. Based on patterns of conservation of nucleotide sequence, the genes encoding Connexin proteins are divided into two families termed the alpha and beta Connexin genes. The carboxy-terminal-most amino acid sequences of alpha Connexins are characterized by multiple distinctive and conserved features. This conservation of organization is consistent with the ability of ACT peptides to form distinctive 3D structures, interact with multiple partnering proteins, mediate interactions with lipids and membranes, interact with nucleic acids including DNA, transit and/or block membrane channels and provide consensus motifs for proteolytic cleavage, protein cross-linking, ADP-ribosylation, glycosylation and phosphorylation. Thus, the provided polypeptide interacts with a domain of a protein that normally mediates the binding of said protein to the carboxy-terminus of an alpha Connexin. For example, nephroblastoma overexpressed protein (NOV) interacts with a Cx43 c-terminal domain⁹. It is considered that this and other proteins interact with the carboxy-terminus of alpha Connexins and further interact with other proteins forming a macromolecular complex. Thus, the provided polypeptide can inhibit the operation of a molecular machine, such as, for example, one involved in regulating the aggregation of Cx43 gap junction channels.

The ACT sequence of the provided polypeptide can be from any alpha Connexin. Thus, the alpha Connexin component of the provided polypeptide can be from a human, murine, bovine, monotrene, marsupial, primate, rodent, cetacean, mammalian, avian, reptilian, amphibian, piscine, chordate, protochordate or other alpha Connexin. Thus, the provided polypeptide can comprise an ACT of a Connexin selected from the group consisting of mouse Connexin 47, human Connexin 47, Human Connexin 46.6, Cow Connexin 46.6, Mouse Connexin 30.2, Rat Connexin 30.2, Human Connexin 31.9, Dog Connexin 31.9, Sheep Connexin 44, Cow Connexin 44, Rat Connexin 33, Mouse Connexin 33, Human Connexin 36, mouse Connexin 36, rat Connexin 36, dog Connexin 36, chick Connexin 36, zebrafish Connexin 36, morone Connexin 35, morone Connexin 35, Cynops Connexin 35, Tetraodon Connexin 36, human Connexin 37, chimp Connexin 37, dog Connexin 37, Cricetulus Connexin 37, Mouse Connexin 37, Mesocricetus Connexin 37, Rat Connexin 37, mouse Connexin 39, rat Connexin 39, human Connexin 40.1, Xenopus Connexin 38, Zebrafish Connexin 39.9, Human Connexin 40, Chimp Connexin 40, dog Connexin 40, cow Connexin 40, mouse Connexin 40, rat Connexin 40, Cricetulus Connexin 40, Chick Connexin 40, human Connexin 43, Cercopithecus Connexin 43, Oryctolagus Connexin 43, Spermophilus Connexin 43, Cricetulus Connexin 43, Phodopus Connexin 43, Rat Connexin 43, Sus Connexin 43, Mesocricetus Connexin 43, Mouse Connexin 43, Cavia Connexin 43, Cow Connexin 43, Erinaceus Connexin 43, Chick Connexin 43, Xenopus Connexin 43, Oryctolagus Connexin 43, Cyprinus Connexin 43, Zebrafish Connexin 43, Danio aequipinnatus Connexin 43, Zebrafish Connexin 43.4, Zebrafish Connexin 44.2, Zebrafish Connexin 44.1, human Connexin 45, chimp Connexin 45, dog Connexin 45, mouse Connexin 45, cow Connexin 45, rat Connexin 45, chick Connexin 45, Tetraodon Connexin 45, chick Connexin 45, human Connexin 46, chimp Connexin 46, mouse Connexin 46, dog Connexin 46, rat Connexin 46, Mesocricetus Connexin 46, Cricetulus Connexin 46, Chick Connexin 56, Zebrafish Connexin 39.9 cow Connexin 49, human Connexin 50, chimp Connexin 50, rat Connexin 50, mouse Connexin 50, dog Connexin 50, sheep Connexin 49, Mesocricetus Connexin 50, Cricetulus Connexin 50, Chick Connexin 50, human Connexin 59, or other alpha Connexin.

The 20-30 carboxy-terminal-most amino acid sequence of alpha Connexins are characterized by a distinctive and conserved organization. This distinctive and conserved organization includes a type II PDZ binding motif (Φ-x-Φ; wherein x=any amino acid and Φ=a Hydrophobic amino acid; e.g., Table 2, BOLD) and proximal to this motif, Proline (P) and/or Glycine (G) hinge residues; a high frequency phospho-Serine(S) and/or phospho-Threonine (T) residues; and a high frequency of positively charged Arginine (R), Lysine (K) and negatively charged Aspartic acid (D) or Glutamic acid (E) amino acids. For many alpha Connexins, the P and G residues occur in clustered motifs proximal to the carboxy-terminal type II PDZ binding motif. The S and T phosphor-amino acids of most alpha Connexins also are typically organized in clustered, repeat-like motifs. This organization is particularly the case for Cx43, where 90% of 20 carboxyl terminal-most amino acids are comprised of the latter seven amino acids. In a further example of the high conservation of the sequence, ACT peptide organization of Cx43 is highly conserved from humans to fish.

Thus, in one aspect, the provided polypeptide comprises one, two, three or all of the amino acid motifs selected from the group consisting of 1) a type II PDZ binding motif, 2) Proline (P) and/or Glycine (G) hinge residues; 3) clusters of phospho-Serine(S) and/or phospho-Threonine (T) residues; and 4) a high frequency of positively charged Arginine (R) and Lysine (K) and negatively charged Aspartic acid (D) and/or Glutamic acid (E) amino acids). In another aspect, the provided polypeptide comprises a type II PDZ binding motif at the carboxy-terminus, Proline (P) and/or Glycine (G) hinge residues proximal to the PDZ binding motif, and positively charged residues (K, R, D, E) proximal to the hinge residues.

PDZ domains were originally identified as conserved sequence elements within the postsynaptic density protein PSD95/SAP90, the Drosophila tumor suppressor dlg-A, and the tight junction protein ZO-1. Although originally referred to as GLGF or DHR motifs, they are now known by an acronym representing these first three PDZ-containing proteins (PSD95/DLG/ZO-1). These 80-90 amino acid sequences have now been identified in well over 75 proteins and are characteristically expressed in multiple copies within a single protein. Thus, in one aspect, the provided polypeptide can inhibit the binding of an alpha Connexin to a protein comprising a PDZ domain. The PDZ domain is a specific type of protein-interaction module that has a structurally well-defined interaction ‘pocket’ that can be filled by a PDZ-binding motif, referred to herein as a “PDZ motif”. PDZ motifs are consensus sequences that are normally, but not always, located at the extreme intracellular carboxyl terminus. Four types of PDZ motifs have been classified: type I (S/T-x-Φ), type II (Φ-x-Φ), type III (Ψ-x-Φ) and type IV (D-x-V), where x is any amino acid, Φ is a hydrophobic residue (V, I, L, A, G, W, C, M, F) and Ψ is a basic, hydrophilic residue (H, R, K)¹⁰. Thus, in one aspect, the provided polypeptide comprises a type II PDZ binding motif.

In some embodiments, the provided polypeptide comprises the c-terminal sequence of human Cx43. Thus, in some embodiments, the polypeptide comprises or consists of the amino acid sequence SEQ ID NO:1 (PSSRASSRPRPDDLEI) or SEQ ID NO:2 (RPRPDDLEI).

When specific proteins are referred to herein, variants, derivatives, and fragments are contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues but can occur at a number of different locations at once; insertions usually will be on the order of about from 1 to 10 amino acid residues. Substitutions, deletions, insertions or any combination thereof may be combined to arrive at a final construct. Substitutional variants are those in which at least one residue has been removed and a different residue inserted in its place.

For example, the replacement of one amino acid residue with another that is biologically and/or chemically similar is known to those skilled in the art as a conservative substitution. For example, a conservative substitution would be replacing one hydrophobic residue for another, or one polar residue for another. Conservatively substituted variations of each explicitly disclosed sequence are included within the polypeptides provided herein.

Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2, 5 or 10 conservative substitutions.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

It is understood that there are numerous amino acid and peptide analogs which can be incorporated into the disclosed compositions. For example, there are numerous D amino acids. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14 (10): 400-403 (1989); Benner, TIB Tech, 12:158-163 (1994); Ibba and Hennecke, Bio/technology, 12:678-682 (1994), all of which are herein incorporated by reference at least for material related to amino acid analogs).

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations (Rizo and Gierasch Ann. Rev. Biochem. 61:387 (1992), incorporated herein by reference).

Thus, the provided polypeptide can comprise a conservative variant of the c-terminus of an alpha Connexin (ACT). It is understood that one way to define any variants, modifications, or derivatives of the disclosed genes and proteins herein is through defining the variants, modification, and derivatives in terms of sequence identity (also referred to herein as homology) to specific known sequences. Specifically disclosed are variants of the nucleic acids and polypeptides herein disclosed which have at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the stated or known sequence. Those of skill in the art readily understand how to determine the sequence identity of two proteins or nucleic acids. For example, the sequence identity can be calculated after aligning the two sequences so that the sequence identity is at its highest level. Another way of calculating sequence identity can be performed by published algorithms.

Thus, the provided polypeptide can comprise an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the c-terminus of an alpha Connexin (ACT). Thus, in one aspect, the provided polypeptide comprises an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to SEQ ID NO: 1, SEQ ID NO: 2, or any sequence provided herein.

In some embodiments, the polypeptide comprises a cellular internalization transporter or sequence. The cellular internalization sequence can be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol). Exemplary cell internalization transporters are provided in Table 1.

TABLE 1
Exemplary cell internalization sequences

| Name | Sequence | SEQ ID NO |
|---|---|---|
| Antp | RQPKIWFPNRRKPWKK | (SEQ ID NO: 7) |
| HIV-Tat | GRKKRRQRPPQ | (SEQ ID NO: 14) |
| Penetratin | RQIKIWFQNRRMKWKK | (SEQ ID NO: 15) |
| Antp-3A | RQIAIWFQNRRMKWAA | (SEQ ID NO: 16) |
| Tat | RKKRRQRRR | (SEQ ID NO: 17) |
| Buforin II | TRSSRAGLQFPVGRVHRLLRK | (SEQ ID NO: 18) |
| Transportan | GWTLNSAGYLLGKINKALAAL | (SEQ ID NO: 19) |
| | AKKIL | |
| model amphipathic | KLALKLALKALKAALKLA | (SEQ ID NO: 20) |
| peptide (MAP) | | |
| K-FGF | AAVALLPAVLLALLAP | (SEQ ID NO: 21) |
| Ku70 | VPMLK-PMLKE | (SEQ ID NO: 22) |
| Prion | MANLGYWLLALFVTMWTDVGL | (SEQ ID NO: 23) |
| | CKKRPKP | |
| pVEC | LLIILRRRIRKQAHAHSK | (SEQ ID NO: 24) |
| Pep-1 | KETWWETWWTEWSQPKKKRKV | (SEQ ID NO: 25) |
| SynB1 | RGGRLSYSRRRFSTSTGR | (SEQ ID NO: 26) |
| Pep-7 | SDLWEMMMVSLACQY | (SEQ ID NO: 27) |
| HN-1 | TSPLNIHNGQKL | (SEQ ID NO: 28) |
| BGSC (Bis- | | |
| Guanidinium- | | |
| Spermidine-Cholesterol) | | |
| BGTC (Bis- | | |
| Guanidinium-Tren- | | |
| Cholesterol) | | |

Any other internalization sequences now known or later identified can be combined with a peptide of the invention.

The provided polypeptide can comprise any ACT sequence (e.g., any of the ACT peptides disclosed herein) in combination with any of the herein provided cell internalization sequences. Examples of said combinations are provided in Table 2. Thus, the provided polypeptide can comprise an Antennapedia sequence comprising amino acid sequence SEQ ID NO: 7. Thus, the provided polypeptide can comprise the amino acid sequence SEQ ID NO:8, SEQ ID NO:9, SEQ ID NO:10, SEQ ID NO: 11, or SEQ ID NO: 12.

TABLE 2
ACT Polypeptides with Cell
Internalization Sequences (CIS)

| CIS/ACT | Sequence | SEQ ID NO |
|---|---|---|
| Antp/ | RQPKIWFPNRRKPWKK | SEQ ID NO: 8 |
| ACT 2 | PSSRASSRASSRPRPDDLEI | |
| Antp/ | RQPKIWFPNRRKPWKK | SEQ ID NO: 9 |
| ACT 1 | RPRPDDLEI | |
| Antp/ | RQPKIWFPNRRKPWKK | SEQ ID NO: 10 |
| ACT 3 | RPRPDDLEV | |
| Antp/ | RQPKIWFPNRRKPWKK | SEQ ID NO: 11 |
| ACT 4 | RPRPDDVPV | |
| Antp/ | RQPKIWFPNRRKPWKK | SEQ ID NO: 12 |
| ACT 5 | KARSDDLSV | |
| HIV-Tat/ | GRKKRRQRPPQ | SEQ ID NO: 56 |
| ACT 1 | RPRPDDLEI | |
| Penetratin/ | RQIKIWFQNRRMKWKK | SEQ ID NO: 57 |
| ACT 1 | RPRPDDLEI | |
| Antp-3A/ | RQIAIWFQNRRMKWAA | SEQ ID NO: 58 |
| ACT 1 | RPRPDDLEI | |
| Tat/ | RKKRRQRRR | SEQ ID NO: 59 |
| ACT1 | RPRPDDLEI | |
| Buforin | TRSSRAGLQFPVGRVHRLLRK | SEQ ID NO: 60 |
| II/ | RPRPDDLEI | |
| ACT 1 | | |
| Transportan/ | GWTLNSAGYLLGKINK | SEQ ID NO: 61 |
| ACT 1 | ALAALAKKIL | |
| | RPRPDDLEI | |
| MAP/ | KLALKLALKALKAALKLA | SEQ ID NO: 62 |
| ACT 1 | RPRPDDLEI | |
| K-FGF/ | AAVALLPAVLLALLAP | SEQ ID NO: 63 |
| ACT 1 | RPRPDDLEI | |
| Ku70/ | VPMLKPMLKE | SEQ ID NO: 64 |
| ACT 1 | RPRPDDLEI | |
| Prion/ | MANLGYWLLALFVTMW | SEQ ID NO: 65 |
| ACT 1 | TDVGLCKKRPKP | |
| | RPRPDDLEI | |
| pVEC/ | LLIILRRRIRKQAHAHSK | SEQ ID NO: 66 |
| ACT 1 | RPRPDDLEI | |
| Pep-1/ | KETWWETWWTEWSQPKKKRKV | SEQ ID NO: 67 |
| ACT 1 | RPRPDDLEI | |
| SynB1/ | RGGRLSYSRRRFSTSTGR | SEQ D NO: 68 |
| ACT 1 | RPRPDDLEI | |
| Pep-7/ | SDLWEMMMVSLACQY | SEQ ID NO: 69 |
| ACT 1 | RPRPDDLEI | |
| HN-1/ | TSPLNIHNGQKL | SEQ ID NO: 70 |
| ACT 1 | RPRPDDLEI | |

Also provided are isolated nucleic acids encoding the polypeptides provided herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Thus, provided is an isolated nucleic acid encoding a polypeptide comprising the amino acid sequence SEQ ID NO:1, SEQ ID NO:2, SEQ ID NO:3, SEQ ID NO:4, SEQ ID NO:5, SEQ ID NO:7, SEQ ID NO:8, SEQ ID NO:9, SEQ ID NO:10, SEQ ID NO:11, or SEQ ID NO:12.

In some embodiments, provided herein is a composition comprising one or more of the herein provided polypeptides, nucleic acids, or vectors in a pharmaceutically acceptable carrier. For example, provided is a composition comprising SEQ ID NO:2 or SEQ ID NO:9 in a pharmaceutically acceptable carrier. In some embodiments, the composition comprises one or more of the herein provided polypeptides encapsulated in a microcarrier. For example, in some embodiments, the composition comprises one or more of the herein provided polypeptides, wherein the polypeptides are in a nanoparticle or exosome.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions provided herein comprising a polypeptide provided herein may be formulated to be administered enterally or parenterally. The compositions provided herein comprising a polypeptide provided herein may be formulated to be administered orally, rectally, vaginally, buccally, sublingually, intramuscular, subcutaneously, intraarterially, transdermally, intraosseously, transmucosally, intravenously, intracerebrally, parenterally, intranasally, intratracheally, by inhalant, or by topical intranasal administration. As used herein, “topical intranasal administration” means delivery of the compositions provided herein comprising a polypeptide provided herein into the nose and nasal passages through one or both of the nares and can comprise delivery by a spraying mechanism or droplet mechanism, or through aerosolization. Administration of the compositions provided herein comprising a polypeptide provided herein by inhalant can be through the nose or mouth via delivery by a spraying or droplet mechanism. Intratracheal administration may include intratracheal injection, instillation, or inhalation. Delivery can also be directly to any area of the respiratory system (e.g., lungs) via intubation, or via a ventilator. Delivery may be via a dry powder inhaler, a metered dose inhaler, a nebulizer (e.g., atomizer jet nebulizer, vibrating mesh nebulizer, or ultrasonic nebulizer), through a mechanical ventilator, or any other means of intranasal, inhalant, intratracheal, or pulmonary administration. Delivery via any of the above administration routes may be in the form of a drug loaded microcarrier formulation, such as nanoparticles or exosomes.

The compositions provided herein comprising an anti-viral agent provided herein may be formulated to be administered enterally or parenterally. The compositions provided herein comprising an anti-viral agent provided herein may be formulated to be administered orally, rectally, vaginally, buccally, sublingually, intramuscular, subcutaneously, intraarterially, transdermally, intraosseously, transmucosally, intravenously, intracerebrally, parenterally, intranasally, intratracheally, by inhalant, or topically. Administration of the compositions provided herein comprising an anti-viral agent provided herein by inhalant can be intravenously. Administration of the compositions provided herein comprising an anti-viral agent provided herein by inhalant can be orally. Further to this embodiment, the anti-vral agent can be a nucleoside analog that possesses a level of oral bioavailability that makes it suitable for formulation into a medicament suitable for oral administration. In some cases, the anti-viral agent can be a nucleoside analog or active metabolite thereof (e.g., remdesivir or active metabolites thereof) that can be delivered orally such as disclosed in US20210379090 and WO/2022/123433A1, each of which is herein incorporated by reference. In some cases, the anti-viral agent can be a nucleoside analog or active metabolite thereof (e.g., remdesivir or active metabolites thereof) in a composition formulated for inhalation such as disclosed in US20210346288, WO/2021/236570A1, WO/2022/016073A1 and US20210353650, each of which is herein incorporated by reference. In some cases, the anti-viral agent can be a nucleoside analog or active metabolite thereof (e.g., remdesivir or active metabolites thereof) in a composition formulated for transmucosal delivery such as disclosed in U.S. Pat. No. 11,020,349, which is herein incorporated by reference. In some cases, the anti-viral agent can be a nucleoside analog or active metabolite thereof (e.g., remdesivir or active metabolites thereof) in a composition formulated for delivery via nebulization such as disclosed in USUS20210252027, which is herein incorporated by reference. Delivery can also be directly to any area of the respiratory system (e.g., lungs) via intubation, or via a ventilator. Delivery may be via a dry powder inhaler, a metered dose inhaler, a nebulizer (e.g., atomizer jet nebulizer, vibrating mesh nebulizer, or ultrasonic nebulizer), through a mechanical ventilator, or any other means of intranasal, inhalant, intratracheal, or pulmonary administration. Delivery via any of the above administration routes may be in the form of a drug loaded microcarrier formulation, such as nanoparticles or exosomes.

In some embodiments, the compositions provided herein comprise drug loaded microcarrier formulations comprising nanoparticles or exosomes. In some embodiments, the size of the nanoparticles is from about 100 nm to about 1000 nm, or about 100 nm to about 500 nm, or about 200 nm to about 250 nm, or about 100 nm to about 200 nm. The drug can be a polypeptide provided herein. The drug can be an anti-viral agent provided herein. The drug can be a polypeptide provided herein and an anti-viral agent provided herein.

In some embodiments, the polypeptide provided herein is administered to the subject at a dose of from about 0.005 mg/kg to about 50 mg/kg. In some embodiments, the polypeptide provided herein is administered to the subject at a dose of about 0.005 mg/kg, about 0.01 mg/kg, about 0.05 mg/kg, about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments, the polypeptide provided herein is administered in a daily dosing regimen. In some embodiments, the polypeptide provided herein is administered to the subject in a formulation comprising about 1 μM to about 10,000 μM of the polypeptide, or about 10 μM to about 9,000 μM, or about 50 μM to about 5,000 μM, or about 100 μM to about 2,000 μM, or about 200 μM to about 2,000 μM, or about 200 μM to about 1,000 μM, or about 50 μM to about 1,500 μM of the polypeptide, or about 100 μM to about 1,000 μM of the polypeptide, or about 500 to about 1,500 μM of the polypeptide. In some embodiments, the polypeptide provided herein is administered to the subject in a formulation comprising about 1 μM, about 5 μM, about 50 μM, about 100 μM, about 150 μM, about 200 μM, about 300 μM, about 400 μM, about 500 μM, about 600 μM, about 700 μM, about 800 M, about 900 μM, about 1,000 μM, about 1,500 μM, about 2,000 μM, about 3,000 μM, about 4,000 μM, about 5,000 μM, about 6,000 μM, about 7,000 μM, about 8,000 μM, about 9,000 μM, or about 10,000 M of the polypeptide. When used in combination with an anti-viral agent (e.g., remdesivir), the polypeptide provided herein can be administered to the subject at a dose of about 0.01 mg/kg or about 0.02 mg/kg.

In some embodiments, the anti-viral agent provided herein (e.g., remdesivir) is administered to the subject at a dose recommended by the manufacturer. In some embodiments, the anti-viral agent provided herein (e.g., a nucleoside analog such as remdesivir or active metabolites thereof or active derivatives thereof) is administered to the subject at a dose of from about 0.1 mg/kg to about 50 mg/kg. In some embodiments, the anti-viral agent provided herein (e.g., a nucleoside analog such as remdesivir or active metabolites thereof or active derivatives thereof) provided herein is administered to the subject at a dose of about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments, the anti-viral agent provided herein (e.g., a nucleoside analog such as remdesivir or active metabolites thereof or active derivatives thereof) is administered in a daily dosing regimen. In some embodiments, the anti-viral agent provided herein (e.g., a nucleoside analog such as remdesivir or active metabolites thereof or active derivatives thereof) is administered to the subject in a formulation comprising about 1 μM to about 10,000 μM of the anti-viral agent provided herein (e.g., a nucleoside analog such as remdesivir or active metabolites thereof or active derivatives thereof), or about 10 μM to about 9,000 μM, or about 50 μM to about 5,000 μM, or about 100 μM to about 2,000 μM, or about 200 μM to about 2,000 μM, or about 200 μM to about 1,000 μM, or about 50 μM to about 1,500 M of the anti-viral agent provided herein (e.g., a nucleoside analog such as remdesivir or active metabolites thereof or active derivatives thereof), or about 100 μM to about 1,000 μM of the anti-viral agent provided herein (e.g., a nucleoside analog such as remdesivir or active metabolites thereof or active derivatives thereof), or about 500 to about 1,500 μM of the anti-viral agent provided herein (e.g., a nucleoside analog such as remdesivir or active metabolites thereof or active derivatives thereof). In some embodiments, the anti-viral agent provided herein (e.g., a nucleoside analog such as remdesivir or active metabolites thereof or active derivatives thereof) provided herein is administered to the subject in a formulation comprising about 1 μM, about 5 μM, about 50 μM, about 100 μM, about 150 μM, about 200 μM, about 300 μM, about 400 μM, about 500 μM, about 600 μM, about 700 μM, about 800 μM, about 900 μM, about 1,000 μM, about 1,500 μM, about 2,000 μM, about 3,000 μM, about 4,000 μM, about 5,000 μM, about 6,000 μM, about 7,000 μM, about 8,000 μM, about 9,000 μM, or about 10,000 UM of the anti-viral agent provided herein (e.g., a nucleoside analog such as remdesivir or active metabolites thereof or active derivatives thereof). When used in combination with the polypeptide provided herein (e.g., aCT1), the anti-viral agent (e.g., a nucleoside analog such as remdesivir or active metabolites thereof or active derivatives thereof), can be administered to the subject at a dose of about 15 mg/kg.

As used herein, “subject” include vertebrates, more specifically a mammal (e.g., a human, horse, pig, rabbit, dog, sheep, goat, non-human primate, cow, cat, guinea pig or rodent), a fish, a bird, a reptile or an amphibian. In some embodiments, the subject is a human subject. The term does not denote a particular age or sex. Thus, adult and newborn subjects, as well as fetuses, whether male or female, are intended to be covered. In some embodiments, a patient refers to a subject afflicted with a disease or disorder. In some embodiments, a patient population refers to a particular, defined set of subjects having a disease or disorder or at risk of developing a particular disease or disorder.

As used herein, “inhibit,” “inhibiting,” and “inhibition” mean to decrease an activity, response, condition, disease, or other biological parameter. This can include, but is not limited to, the complete loss of activity, response, condition, or disease. Thus, the reduction can be a 10, 20, 30, 40, 50, 60, 70, 80, 90, 100%, or any amount of reduction in between as compared to native or control levels.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

By “treat” or “treatment” is meant a method of reducing the effects of a disease or condition. Treatment can also refer to a method of reducing the underlying cause of the disease or condition itself rather than just the symptoms. The treatment can be any reduction from native levels and/or any improvement of clinical signs of the disease and/or any increase in survival or function; and can be but is not limited to the complete ablation of the disease, condition, or the symptoms of the disease or condition. For example, a disclosed method for treating ARDS is considered to be a treatment if there is a reduction in one or more symptoms of the disease or if there is an improvement in the condition of the subject when compared to native levels in the same subject or control subjects. Thus, the reduction or improvement can be a 10, 20, 30, 40, 50, 60, 70, 80, 90, 100%, or any amount of reduction in between as compared to native or control levels. By “prevent” or “prevention” and the like is meant a method of preventing, or reducing the most severe complications of, a viral respiratory disease or disorder.

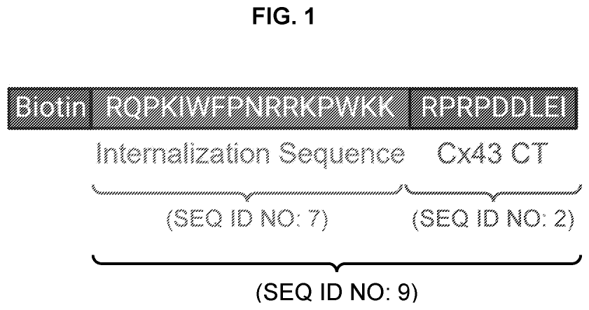

The present disclosure provides a method of use of antiviral agents that inhibit viral replication (e.g., nucleoside analogs) in combination with use of a class of bioengineered Connexin43-based peptides that show therapeutic promise in the field of tissue engineering and regenerative medicine, including the injured lung epithelium¹²-14 for treating respiratory disorders or conditions such as, for example, ARDS, ALI, pneumonia, fibrosis, acute lung injury, COPD, alveolar hemorrhage, pulmonary edema and lung inflammation. An exemplary anti-viral agent is an inhibitor of viral replication. An exemplary anti-viral agent is an inhibitor of an RNA dependent, RNA polymerase. An exemplary anti-viral agent is a nucleoside analog or nucleotide analog. An exemplary anti-viral agent is an active metabolite of a nucleoside analog prodrug or a derivative of a nucleoside analog prodrug. An exemplary anti-viral agent is an adenosine analog, an active metabolite of an adenosine analog prodrug, or a derivative of an adenosine analog. An exemplary anti-viral agent is remdesivir, an active metabolite thereof or a derivative (e.g., ester prodrug) thereof. An exemplary anti-viral agent is a derivative (e.g., ester prodrug) of the parent nucleoside of remdesivir (i.e., GS-441524), such as, for example, ATV006, GS-621763, and VV116. An exemplary anti-viral agent is a cytidine analog, an active metabolite of a cytidine analog prodrug, or a derivative of a cytidine analog. An exemplary anti-viral agent is an guanosine analog, an active metabolite of an guanosine analog prodrug, or a derivative of an guanosine analog. An exemplary anti-viral agent is a uridine analog, an active metabolite of a uridine analog prodrug, or a derivative of a uridine analog. An exemplary anti-viral agent is a thymidine analog, an active metabolite of a thymidine analog prodrug, or a derivative of a thymidine analog. The exemplary peptide aCT1 (FIG. 1) is a 25 aa peptide (3597.33 Da) that has a compact 2-domain design based on linkage of an Antennapedia cell internalization domain (1-16aa; RQPKIWFPNRRKPWKK; SEQ ID NO: 7) to the C-terminal PDZ binding domain of the transmembrane gap junction protein Cx43 (17-25aa; RPRPDDLEI; SEQ ID NO:2)¹⁴. Accordingly, the full aCT1 sequence is RQPKIWFPNRRKPWKK RPRPDDLEI (SEQ ID NO: 9). aCT1 and related peptides interact with known binding partners of Cx43, including the tight junction scaffolding protein zonula occludens 1 (ZO-1)^{15, 16} aCT1 was developed as a molecular tool to inhibit ZO-1 binding to the Cx43 C-terminus by binding to the PDZ2 motif on ZO-1 itself¹⁶. aCT1 has also been shown to interact directly with Cx43's C-terminal domain¹⁷ and may have other binding partners such as CCN3⁹, 14-3-3 proteins¹⁸, SH3-mediated interactions¹⁹, and various protein kinases²⁰. Cx43 binds the PDZ2 domain of ZO-1, which regulates the size and stability of gap junctions by altering the relative proportion of hemichannels to gap junction channels. When aCT1 disrupts Cx43/ZO-1 interactions, this causes a shift in Cx43 from nonjunctional hemichannels to gap junctional complexes. Collectively, this ZO-1 mediated translocation of hemichannels into gap junctions simultaneously enhances gap junction intercellular communication (GJIC) while reducing hemichannel activity, such as release of ATP or other small molecules into the extracellular space that can drive an inflammatory response¹⁵. aCT1 has been shown to affect the connexin life cycle in several ways, from increasing gap junction plaque size to influencing kinase activity to regulating localization of Cx43^{16, 20-23}.

aCT1's small, stable, soluble design facilitates direct translocation into cells without requirement for potentially toxic excipient compounds for intracellular drug delivery. aCT1 stabilizes intercellular junctions, reduces the release of proinflammatory cytokines, and promotes an effective epithelial response to injury.

aCT1 treatment reduces inflammation at the tissue level. For example, in ocular corneal injury models, aCT1 treatment has reduced inflammatory signaling molecules such as IL-6, IL-1β, TNFα, Cox-2, MMP-9, and VEGF. Activated neutrophils have been shown to release ATP via Cx43 hemichannels, and Cx43-dependent release of ATP has been shown to recruit macrophages^{25, 26}. Notably, inhibiting Cx43 hemichannel mediated ATP release has been shown to reduce the early inflammatory response²⁷, and aCT1 has been shown to recruit more hemichannels into gap junctions, thereby depleting the hemichannel pool. These data support a role for reduction in hemichannel-mediated ATP release as a trigger of downstream events that culminate in the observed reduction in inflammatory cell infiltration with aCT1 treatment.

Tight junctions form the intercellular barrier between epithelial and endothelial cells, controlling paracellular permeability of water, ions, and macromolecules. Tight junction assembly is dependent upon the oligomerization of integral membrane proteins known as claudins into tight junction strands, to form a barrier between cells⁴¹. Claudin assembly at the cell membrane is directed by ZO-1, a cytosolic scaffold protein that anchors tight junctional transmembrane proteins to the cellular cytoskeleton. ZO-1 binds claudin C-termini and promotes claudin oligomerization into tight junction strands, forming a seal between neighboring cells⁴². In addition, ZO-1 interacts with transmembrane protein occludin, which contributes to tight junction stability and optimal barrier function⁴³. ZO-1 also regulates the assembly of adherens and gap junctions, supporting a general role for ZO-1 in intercellular adhesion and junctional stabilityl^{5, 44, 45}. When aCT1 binds ZO-1, ZO-1's claudin selective PDZ1 domain may be exposed and therefore able to interact with claudins⁴⁶. aCT1 stabilizes ZO-1 at the plasma membrane, allowing claudins to oligomerize into tight junction strands, thus preventing tight junction degradation in response to injury and supporting accelerated re-establishment of cell barriers⁴⁷. aCT1 is able to promote cell barrier integrity and reduce edema across several organ systems, supporting a role for aCT1 in the regulation of tight junctions' response to injury.

In addition to effects on inflammation, cell barrier integrity, and edema, aCT1 has demonstrated an ability to promote re-epithelialization and accelerate wound healing. aCT1 has been shown to alter fibroblast migration, collagen fiber type, and collagen fiber deposition pattern in dermal and surgical models^{48, 49}.

Without wishing to be bound by theory, aCT1 directly targets and repairs the damaged cells lining airspaces and vasculature in the injured lung, thus restoring barrier integrity and directly addressing the cause of ARDS. While antiviral therapies in development for viruses such as SARS-CoV-2 can limit viral replication and accelerate viral clearance, these therapies will not address the need for rapid repair of the pulmonary epithelium in critically ill patients to prevent progression to severe pneumonia and then to ARDS.

Preclinical and clinical studies have demonstrated the safety and efficacy of the active pharmaceutical ingredient aCT1 peptide. Intravenous administration (bolus) of aCT1 in rats was well tolerated and a 5 mg/kg dosing level was established as the no-observable-adverse-effect level (NOAEL). Clinical signs were only observed at aCT1 administration levels that are well above the therapeutic range of efficacy, following intravenous administration of aCT1 at ≥10 mg/kg (Maximum Tolerated Dose). The results of these studies uniformly support aCT1 tolerability when delivered locally and systemically via various routes.

Clinical trials have been undertaken to assess safety of topical delivery of aCT1. These clinical trials have included over 482 human subjects with no drug related adverse events. These trials have demonstrated the efficacy and safety of local delivery of aCT1 to both acute and chronic skin injuries^{12, 51-53}. Furthermore, aCT1 was not immunogenic in any preclinical study or clinical trial (i.e., no anti-aCT1 antibodies were detected). The half-life of aCT1 in human blood is 15-20 mins (ex vivo studies) and pharmacokinetic studies included in clinical trials indicate no systemic exposure, underscoring local activity. Clinical trials to date have evaluated the safety, pharmacokinetics and immunogenicity of aCT1 when applied topically in maximal clinical use conditions, with favorable results.

The present disclosure provides experiments carried out to determine if an anti-viral agent (e.g., nucleotide analog such as remdesivir) used in combination with an alpha Connexin polypeptide is useful in the treatment of COVID-19 patients to resolve symptoms of SARS-CoV-2 induced lung injury, thus reducing incidence and severity of ARDS. Surprisingly, the inventors of the present application found that the polypeptide aCT1, when administered to the lungs via intranasal or aerosolized delivery, is highly effective in treating, mitigating the symptoms of, and improving clinical outcomes associated with acute lung injury and ARDS when used in combination with administration of a nucleotide analog anti-viral agent (e.g., remdesivir).

The present disclosure is further illustrated by reference to the following Examples. However, it should be noted that these Examples, like the embodiments described above, are illustrative and are not to be construed as restricting the scope of the disclosure in any way.

### EXAMPLES

### Example 1. Efficacy of aCT1 Peptide in Treating Lung Injury

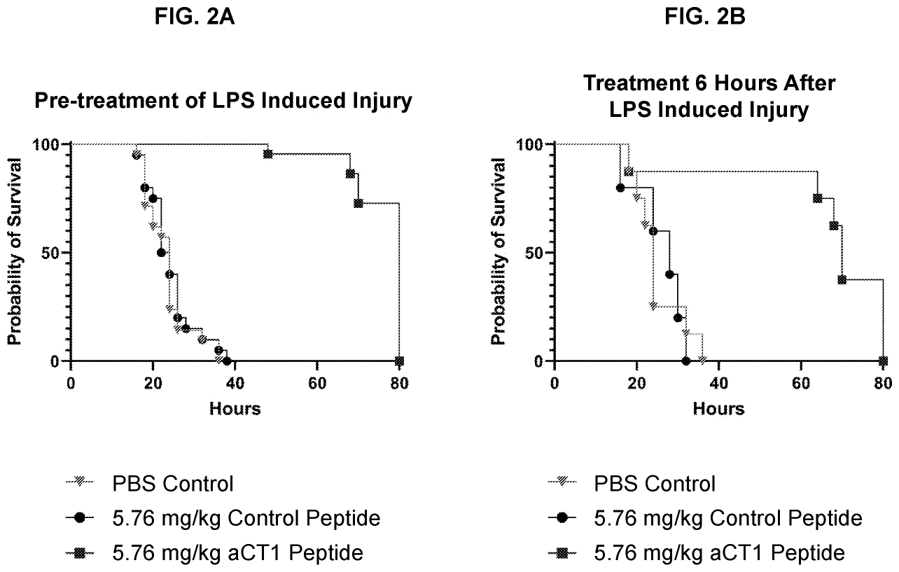

Studies were undertaken to assess whether delivery of aCT1 to the respiratory tract in animal models of ARDS would reduce epithelial and endothelial breakdown, thereby preserving the air-liquid barrier while preventing fluid accumulation and reducing immune cell infiltration, resulting in the amelioration of ARDS pathology and preserving lung function to improve survival. Using a well characterized mouse model of ARDS, we investigated whether aCT1 applied directly to the lungs could inhibit severe endotoxin induced lung injury. C57BL/6 mice were intranasally challenged with a lethal dose of lipopolysaccharide (LPS) and were treated with aCT1 immediately prior to LPS administration (FIG. 2A), or six (6) hours post LPS exposure (FIG. 2B). Both aCT1 pre or post treatment significantly improved survival in response to a lethal dose of LPS.

### Example 2. Efficacy of aCT1 Peptide in a Model of Sepsis

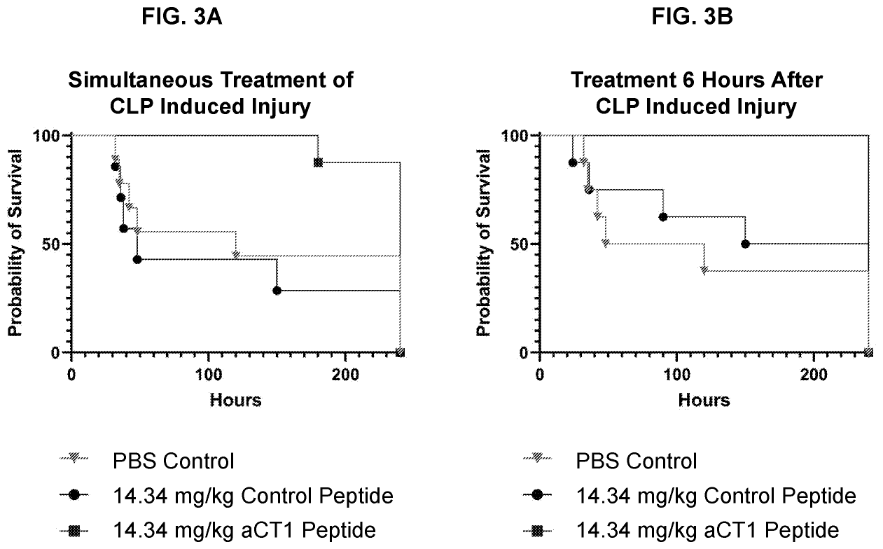

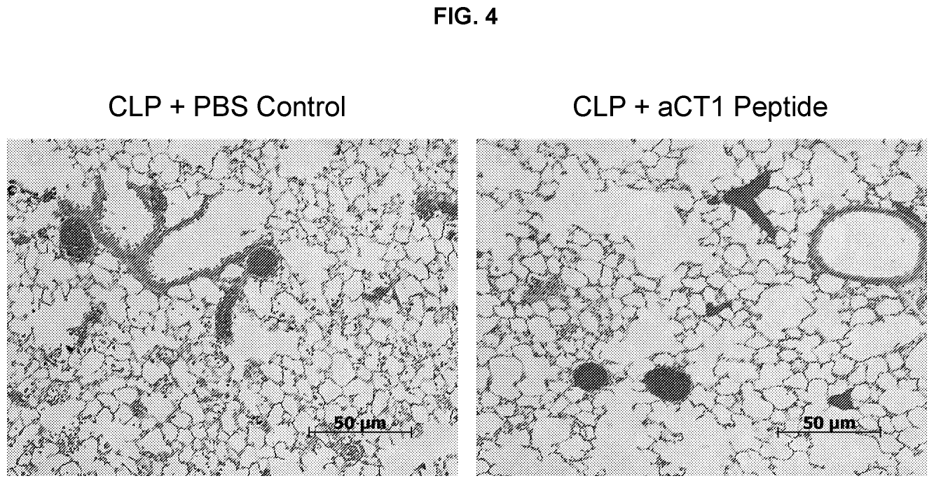

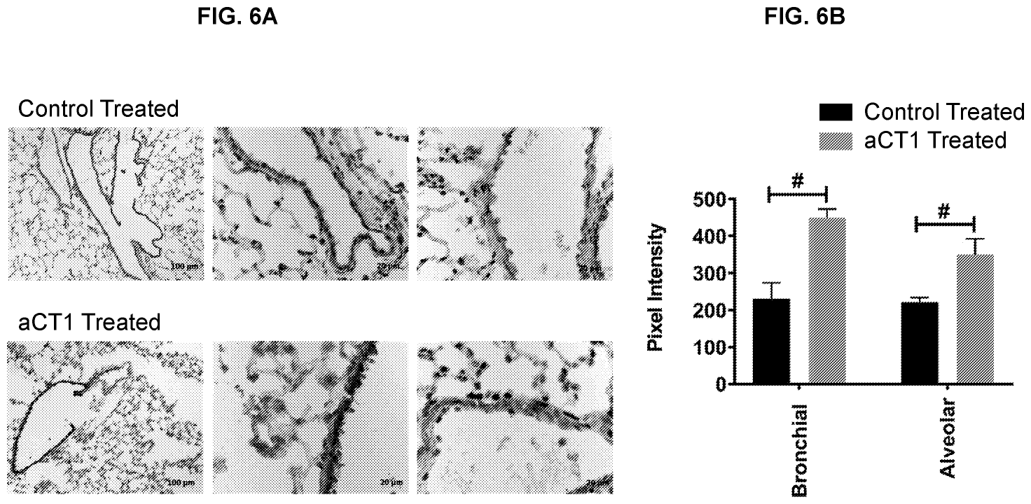

Sepsis is one of the most common causes of ARDS, causing diffuse inflammation in the lung and injury to the airway epithelium. In a well characterized cecal ligation and puncture (CLP) mouse model of sepsis, treatment with intravenously administered aCT1 immediately after injury (FIG. 3A) or 6 hours post CLP procedure (FIG. 3B) prolonged survival. Additionally, administration of aCT1 (14.34 mg/kg) immediately after CLP procedure significantly decreased immune cell infiltration and alveolar edema (FIG. 4).

### Example 3. Safety and Localization of aCT1 in Lung Tissue

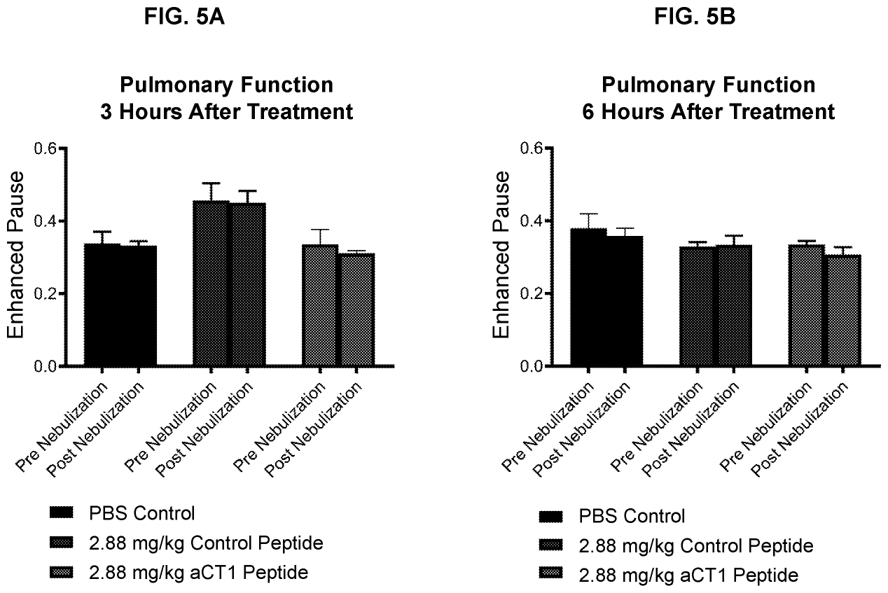

Aerosolized delivery of aCT1 to the lung is readily applicable in the hospital setting and would easily integrate into existing treatment paradigms for patients suffering from viral respiratory infections. To test the ability of aCT1 peptide to reach alveoli and airways, healthy mice were exposed to 2.88 mg/kg aerosolized aCT1. aCT1 was detected in bronchial and alveolar epithelial cells and the endothelial cells of the microvasculature. Critically, aerosolized administration of up to 2.88 mg/kg aCT1 had no effect on enhanced pause (Penh), a general measure of pulmonary function three (3) hours after nebulization (FIG. 5A) or six (6) hours after nebulization (FIG. 5B). Delivery of up to 2.88 mg/kg aCT1 was not associated with alteration in lung function nor caused any visual signs of distress or labored breathing in healthy animals. FIG. 6A shows the localization of aCT1 staining 6 hours after nebulized delivery of aCT1 vs. saline control, in respiratory epithelial cells and endothelial cells of the microvasculature. FIG. 6B provides quantification of stained Cx43 phosphorylated at S368 in bronchial and alveolar cells of animals that received aCT1 or saline (PBS) control.

Taken together, the results of the studies indicate an alpha Connexin polypeptide (e.g., aCT1) prolongs survival in response to severe acute lung injuries, reduces inflammatory cell infiltration and edema, and has no adverse effect on pulmonary function in vivo. Treatment with such alpha Connexin polypeptides offers a unique therapeutic opportunity to modulate the lung injury response following viral respiratory infection by stabilizing intercellular junctions and tempering Cx43 hemichannel activity. Without wishing to be bound by theory, in the virally damaged lung, the alpha Connexin polypeptide will decrease lung inflammation, preserve the air-liquid barrier, and reduce injury spread. This surprisingly effective therapeutic benefit of aCT1 will translate in the clinic as a reduction in the severity of virus induced lung injury, promoting lung function and accelerating healing, thus preventing pneumonia progression and ARDS.

### Example 4. Evaluation of Therapeutic Efficacy of aCT1 Peptide in Preventing SARS-CoV-2 Induced Lung Injury

Animal Model of SARS-CoV-2: The MERS-COV and SARS-COV outbreaks of the 2000s highlighted the utility of non-human primates (NHPs) as the premiere translationally relevant species for modeling the human course of coronavirus diseases. The advancement and development of therapeutics to treat these severe respiratory infections in humans relied on NHPs; these animals are naturally susceptible to SARS and MERS-COV infection and share many physiological similarities with humans. African green monkeys develop hallmarks of the severe lung injury induced by SARS- and MERS-COVs, including clinically significant lesions to the alveolar and bronchiolar walls of the lung, inflammatory infiltration, and flooding of the airspaces leading to pulmonary edema^{54, 55}. Given the genetic and pathological similarities of SARS-COV and SARS-CoV-2, the animal model of SARS-COV in African green monkeys serves as a foundation for development of an animal model of SARS-CoV-2 in NHP^{4, 55, 56}. Accordingly, studies will be undertaken to validate the efficacy and safety of aCT1 peptide as a therapeutic for lung injury, for example, SARS-CoV-2 induced lung injury.

A dose-ranging study using the African green monkey model of SARS-CoV-2 infection is designed to validate the safety and efficacy of aCT1 in a translationally relevant animal model and enable rapid progression to clinical evaluation in COVID-19 positive subjects. Monkeys are inoculated with SARS-CoV-2 and assigned to treatment groups. Efficacy of aCT1 is tested in a prophylactic treatment paradigm with administration at/01U the onset of infection (e.g., 1, 2, 3, 4, 5, 6, or 7 days post inoculation) and in a therapeutic treatment paradigm with administration beginning at the onset of symptoms (e.g., 7, 8, 9, 10, 11, 12, 13, 14, or more days post inoculation). A high dose and low dose of aCT1 are tested in each treatment paradigm. To confirm safety of lung delivery and complement aCT1's existing toxicology package, a group of monkeys (male and female) receive daily aCT1 administered intranasally, without viral challenge. Exemplary treatment groups are provided in Table 3. Treatment is administered daily for the study duration. At 21 days post-inoculation, animals are euthanized and necropsied. Lung tissue is collected for quantitative analysis of viral RNA levels by qRT-PCR to confirm viral infection and quantify tissue burden.

TABLE 3
Exemplary Treatment Groups

| Group | Animal | Viral | | Treatment |
|---|---|---|---|---|
| Assignment | Number | Challenge | Treatment | Start |
| Group 1 | 4 (2M, 2F) | None | aCT1 (high | 7 day |
| | | | dose) | post inoc. |
| Group 2 | 8 (4 M, 4 F) | SARS-CoV-2 | Vehicle | 7 day |
| | | | | post inoc. |
| Group 3 | 8 (4 M, 4 F) | SARS-CoV-2 | aCT1 (high | 7 day |
| | | | dose) | post inoc. |
| Group 4 | 8 (4 M, 4 F) | SARS-CoV-2 | aCT1 (low | 7 day |
| | | | dose) | post inoc. |
| Group 5 | 8 (4 M, 4 F) | SARS-CoV-2 | aCT1 (High | 14 d |
| | | | dose) | post inoc. |
| Group 6 | 8 (4 M, 4 F) | SARS-CoV-2 | aCT1 (low | 14 d |
| | | | dose) | post inoc. |

Clinical Observations: Beginning on the day of inoculation (day 0), all animals will be observed for signs of disease and clinical scores, including scoring of respiratory signs. Clinical examinations are performed on day 0, 7, and 14 with measurements of respiration rate of anesthetized animals.

Pulmonary Function: The therapeutic effect of aCT1 on pulmonary function will be assessed using real time plethysmography to measure tidal volume and respiratory rate. These measurements and blood oxygenation (pulse oximetry) will be performed on Day 21 prior to euthanasia. Demonstration of aCT1 efficacy in preserving lung function directly translates to decreased need for mechanical ventilation and improved survival.

Necropsy and Histopathology: Organs will be examined grossly and findings will be documented by a veterinary pathologist. Lung tissue samples will be fixed, sectioned, and stained for histopathological scoring. Stained slides will be analyzed and scored for inflammatory infiltrates, lung lesions, thickening of the alveolar septae, and alveolar edema by a veterinary pathologist. Improvement in lung histopathology scores with aCT1 treatment provide evidence of aCT1's ability to prevent lung injury and inflammation, thus limiting severity of SARS-CoV-2 associated lung injury.

Safety Analyses: Adverse events will be documented and designated as treatment related or non-treatment related by veterinary assessment. Adverse events will be compared between all treatment groups. Data collected from non-viral challenged monkeys receiving daily intranasal aCT1 will be used to confirm a lack of any systemic effects of aCT1 as well as the safety of delivering aCT1 to the lung.

The goal of the study will be to show that aCT1 is effective in safe in preventing and treating lung injury and inflammation associated with viral infection.

### Example 5. ACT1 Efficacy in Lung Injury

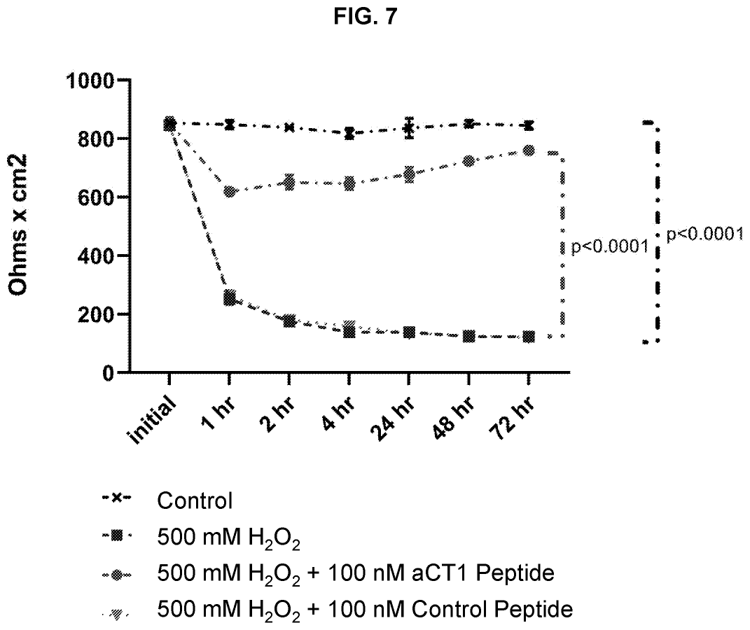

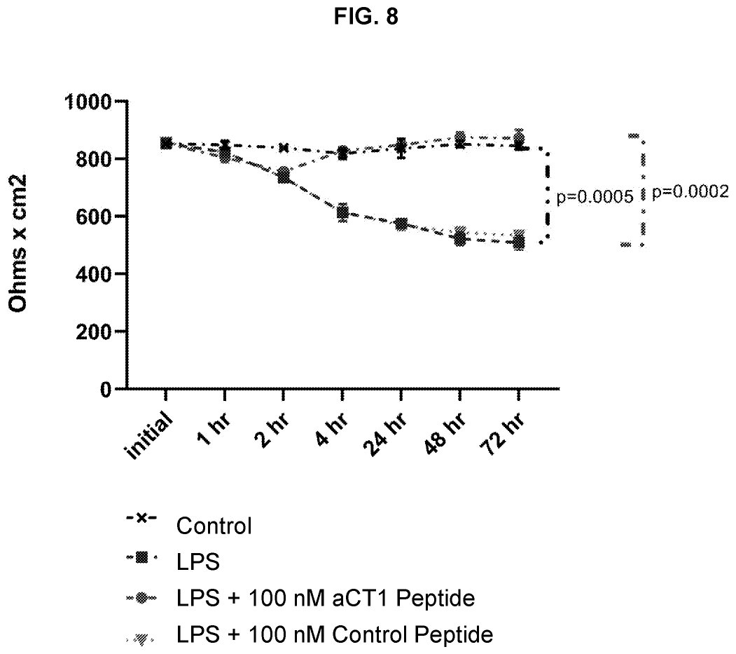

To determine if aCT1 treatment preserves junctional integrity in human lung cells, human bronchial epithelial cells (NHBEs) were grown as confluent monolayers on Transwell inserts and trans-epithelial electrical resistance (TER) recorded. TER readings measure electrical resistance across a cell monolayer and provide a quantitative metric of intercellular junction integrity. Treatment with aCT1 stabilized intercellular junctions following exposure to 500 mM H₂O₂, demonstrating the ability of aCT1 to protect epithelial barrier integrity in response to oxidative stress in human lung cells (FIG. 7). aCT1 pretreatment of NHBEs also stabilized barrier integrity in response to LPS endotoxin exposure, as measured by TER, while untreated cells demonstrated decreased electrical conductivity indicative of junctional breakdown (FIG. 8). Taken together, the studies showed that aCT1 peptide stabilizes junctional barriers in human lung cells when administered prior to various insults.

### Example 6. Evaluation of Therapeutic Efficacy of aCT1 Peptide Via Nose-Only Inhalation in the SARS-CoV-2 Model

### Objective

The objective of this study was to evaluate the efficacy of aCT1 peptide when administered through inhalation routes and in combination with an inhibitor of viral replication (i.e., remdesivir) in a hamster SARS-CoV-2 challenge model.

### BACKGROUND

In the current coronavirus disease (COVID-19) pandemic, it is imperative to identify effective treatments against the SARS-CoV-2 virus as rapidly as possible. Prior to use in humans, potential therapeutic efficacy needs to be shown in germane animal models. It is most imperative that these models provide predictive value for the therapy when it is used to treat SARS-CoV-2 in humans. One model that has shown to be permissive to SARS-CoV-2 infection and demonstrates mild-to-moderate disease is the Syrian hamster⁵⁷. The study proposed here took advantage of this hamster model and was used to evaluate the efficacy of aCT1 peptide treatment in combination with a standard of care antiviral (remdesivir) designed to reduce viral loads and consequent disease burden.

### Methods

The experimental design for this study is shown in Table 4. In brief, after quarantine all animals were assigned to the treatment groups below. Animals in Groups 2-7 underwent viral challenge with SARS-CoV-2 by intranasal instillation (IN) on Day 0. Group 2 was inoculated with SARS-CoV-2 as the challenge control and received no treatment. Group 3 received remdesivir via intraperitoneal injection (IP) on Days 1-4. Groups 4 and 5 received aCT1 via nose-only inhalation on Days 1, 2, 3, and 5 post challenge. Groups 6 and 7 received aCT1 via nose-only inhalation on Days 1, 2, 3, and 5 post challenge and remdesivir via IP injection on Days 1-4. Viral infection was confirmed by viral titering of lung tissues using qRT-PCR. Animals had daily observations and body weights beginning Day-3 through necropsy. Half the animals/sex in each group was euthanized on study Day 4 (prior to treatments) and the remaining animals were euthanized on Study Day 7.

TABLE 4
Experimental Design

| | Animal | | | | | |
|---|---|---|---|---|---|---|
| Group | Number | | | | | |
| Assignment | (N/sex) | SARS-CoV-2 | TA Route | TA Dose | | Necropsy |
| 1 - aCT1 High | 8 (4M/4F) | − | Nose-only | 20 | μg/kg* | Day 4 and |
| Dose TA | | | inhalation | | | Day 7 |
| Control (no | | | | | | |
| challenge) | | | | | | |
| 2 - Challenge | 16 (8M/8F) | + | n/a | n/a | | |
| Control | | | | | | |
| 3 - Remdesivir | 16 (8M/8F) | + | IP | 15 | mg/kg | |
| Control | | | | | | |
| 4 - aCT1 Low | 16 (8M/8F) | + | Nose-only | 10 | μg/kg* | |
| Dose | | | inhalation | | | |
| 5 - aCT1 High | 16 (8M/8F) | + | Nose-only | 20 | μg/kg* | |
| Dose | | | inhalation | | | |

| | | | | |
|---|---|---|---|---|
| 6 - aCT1 Low | 16 (8M/8F) | + | Nose-only | 10 μg/kg* + |
| Dose + | | | inhalation + | 15 mg/kg |
| Remdesivir | | | IP | |
| 7 - aCT1 High | 16 (8M/8F) | + | Nose-only | 20 μg/kg* + |
| Dose + | | | inhalation + | 15 mg/kg |
| Remdesivir | | | IP | |

*targeted deposited dose.
*The actual estimated deposited doses were expected to be about 2x higher

### Treatment Administration

Groups 3, 6, and 7 were administered remdesivir at 5 mg/mL by IP administration See Table 4 for doses.

Lyophilized aCT1 was reconstituted with sterile saline prior to dose administration.

Inhalation exposures for Groups 1, 4, 5, 6, and 7 was conducted using an Aerogen Solo nebulizer and rodent nose-only exposure chamber. Exposure oxygen levels (%) were monitored throughout the exposure.

Total aerosol concentration in test atmospheres was determined by gravimetric analysis of filter samples (47-mm fiber film filters, Type GF/A, GE Whatman, Inc.) collected every 5 minutes directly from a nose-only exposure port during exposures at a target nominal flow at 0.3±0.1 L/min. After collection, the filters were removed from the filter holders and weighed. This data was used to calculate the total (mass) aerosol concentration in the exposure atmosphere.

Particle size distribution was measured at 1 time in the study (once for each exposure) at the breathing zone using an InTox Mercer cascade impactor (InTox Products, Moriarity, NM) operated at a nominal flow rate of 2.0 L/min.

Respiratory minute volume (RMV; liters per min) was calculated using the following allometric equation: RMV=0.608×BW\hat{ }(0.852), where BW is the animal's body weight in kilograms⁵⁸.

The estimated dose is calculated using the following formula: Dose=(C×RMV×T×DF)/BW.

Where C is the average concentration of the test article (TA) in the exposure atmosphere (mg/L), T (min) is exposure time, and the deposition fraction (DF) is assumed to be 100% for the presented dose and 10% for the pulmonary deposited dose⁵⁹.

### Clinical Observations

Detailed observations were performed twice a day (a.m. and p.m.), with special attention to normal behavior and continuing until the end of the study, or until moribund or found dead.

### Body Weights

A pre-study weight of all animals was collected for randomization. The animals were also weighed daily beginning on Day −3 for the duration of the study.

Scheduled necropsies included sample collection and processing according to Table 5. Necropsies included a terminal bodyweight, whole lung weight, and lung samples for various analyses (with sample weight). Sample collection, processing and gross necropsy observations of the lung were recorded and documented lesion location(s), size, shape, color, consistency, and number.

TABLE 5
Tissue Collection

| | | | | | Tissue | |
|---|---|---|---|---|---|---|
| | | | | | frozen for | |
| | | | RT-PCR | Possible | possible | Tissues |
| | Gross | Organ | (genomic | TCID 50 | future | fixed for |
| Tissue | Pathology | Weights | and sgm) | Analysis | analysis | pathology |
| Lung | x | x | | | | |
| a) Left | | x | x | x | x | |
| Lung lobe | | | | | | |
| b) right | | x | | | | x |
| cranial lung | | | | | | |
| lobe | | | | | | |
| c) right | | x | | | | x |
| middle | | | | | | |
| lung lobe | | | | | | |
| d) right | | x | | | | x |
| caudal lung | | | | | | |
| lobe | | | | | | |
| e) | | x | | | | x |
| infracardiac | | | | | | |
| lung lobe | | | | | | |
| Gross | x | | | | | x |
| Lesions^{A} | | | | | | |

^{A}Any gross lesions were sampled and preserved for potential histopathologic analysis.

### Microscopic Evaluation

After formalin fixation, lung tissue was trimmed, paraffin embedded, and taken to slide. Slides were stained with hematoxylin and eosin (H&E).

Histologic lesions were scored based on the severity and distribution of pathology by a veterinary pathologist. Histopathologic changes in the examined tissues were graded semi-quantitatively by a single pathologist on a scale of 0-5.

### Results and Conclusions

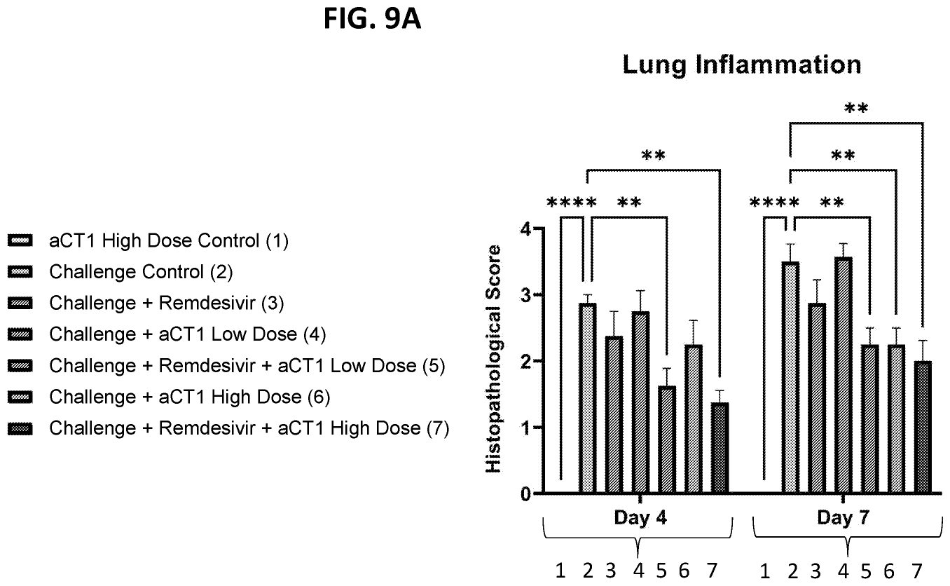

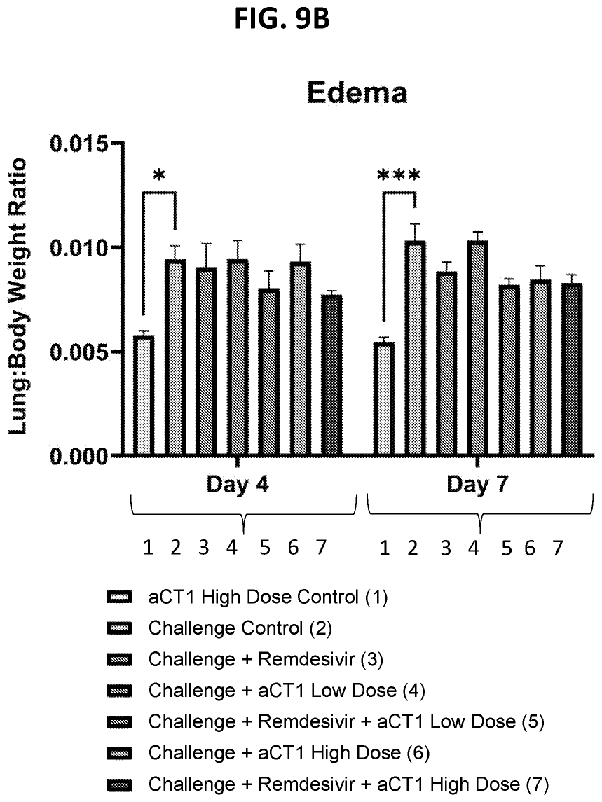

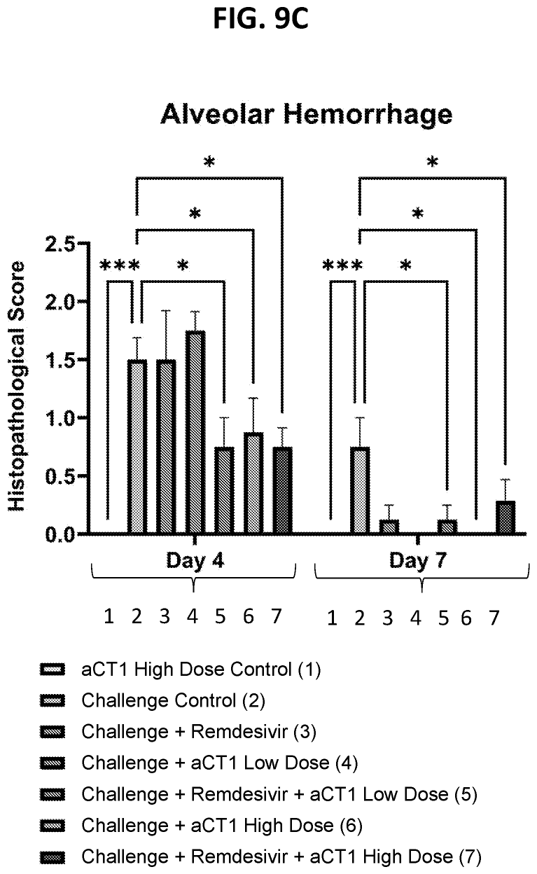

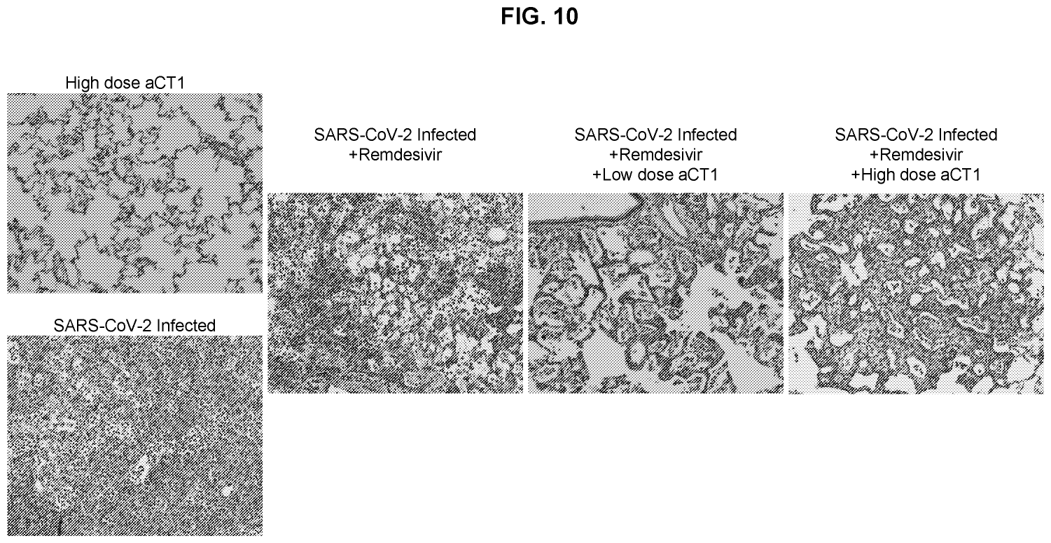

Aerosolized aCT1 in combination with remdesivir mitigated inflammatory cell infiltration into the lung tissue (see FIG. 9A) in SARS-CoV-2 infected hamsters. In animals treated with low dose aCT1+remdesivir, a reduction in inflammatory cell infiltration beyond the additive effects of remdesivir alone and low dose aCT1 alone was observed, supporting an unexpectedly synergistic effect. Treatment with aCT1 and remdesivir also prevented an increase in lung:body weight ratio, which is indicative of pulmonary edema (see FIG. 9B). Again, animals treated with low dose aCT1+remdesivir demonstrated a reduction in lung:body weight ratio beyond the additive effects of remdesivir alone and low dose aCT1 alone on day 4 post challenge. In addition, histopathological analysis of lung tissue showed that animals treated with low dose aCT1+remdesivir had reduced alveolar hemorrhage beyond the additive effects of remdesivir alone and low dose aCT1 alone on day 4 post challenge (see FIG. 9C). These effects were evidenced in the lung tissue slices in FIG. 10. Overall, the hamster model of COVID-19 showed that aCT1+remdesivir prevented SARS-CoV-2 induced pulmonary edema, reduced inflammation and reduced alveolar hemorrhage more effectively than remdesivir alone.

### Example 7. Efficacy of aCT1 Peptide in a Model of ARDS

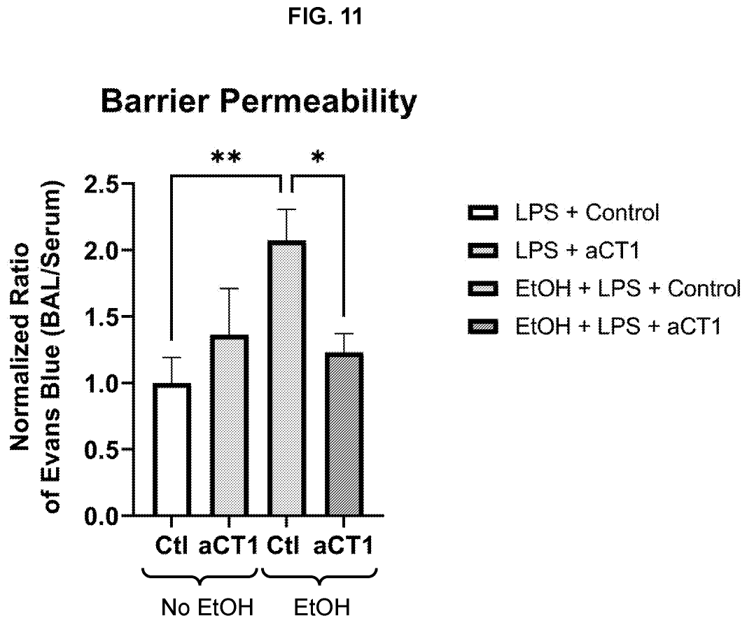

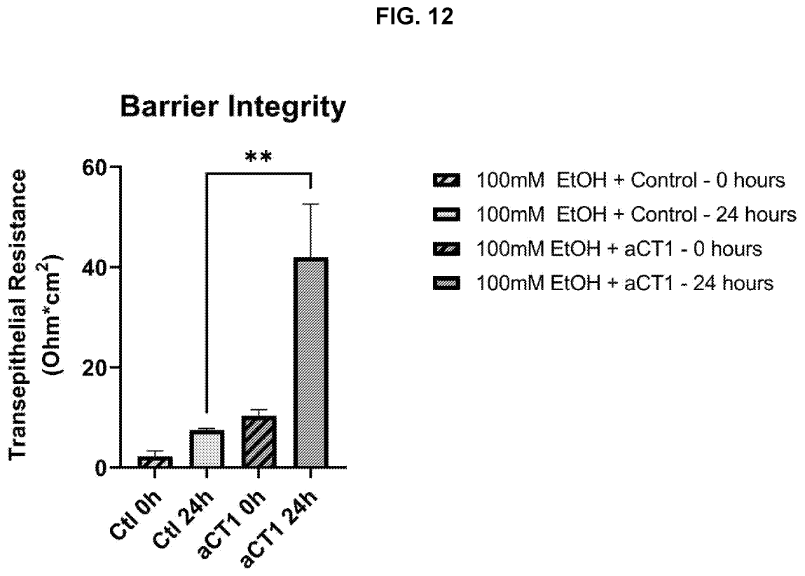

Alcoholic lung syndrome is one of the most common causes of ARDS, chronically compromising the pulmonary barrier function. In a well characterized mouse model of ARDS in alcoholic mice, aerosolized aCT1 prevented LPS-induced permeability in the lung in alcoholic mice (see FIG. 11). In C57BL/6 mice fed ethanol at increasing concentrations and then maintained on 20% ethanol in drinking water for an additional 10 weeks, mice treated with 5 mg/kg aerosolized aCT1 peptide 1 hour after intratracheal instillation of 5 mg/kg LPS to induce lung injury showed reduced pulmonary barrier permeability back to non-alcoholic levels. Moreover, in vitro alcohol treated primary human lung cells showed improved electrical resistance, indicative of improved barrier function, when treated with aCT1 peptide (see FIG. 12).

Overall, the Examples presented herein demonstrate that aCT1 peptide administered locally to the lung demonstrated preliminary efficacy in several models of acute lung injury, highlighting therapeutic potential in acute respiratory distress syndrome, pneumonia, and many other conditions with unmet clinical needs.

### REFERENCES

1. Administration, U.S.F.a.D., Vol. 2023 (2022).
2. Koval, M. Claudin Heterogeneity and Control of Lung Tight Junctions. Annual Review of Physiology 75, 551-567 (2013).
3. Lin, L., Lu, L., Cao, W. & Li, T. Hypothesis for potential pathogenesis of SARS-CoV-2 infection—a review of immune changes in patients with viral pneumonia. Emerg Microbes Infect, 1-14 (2020).
4. Khan, S. et al. The emergence of a novel coronavirus (SARS-CoV-2), their biology and therapeutic options. J Clin Microbiol (2020).
5. Xu, Z. et al. Pathological findings of COVID-19 associated with acute respiratory distress syndrome. Lancet Respir Med (2020).
6. Yang, X. et al. Clinical course and outcomes of critically ill patients with SARS-CoV-2 pneumonia in Wuhan, China: a single-centered, retrospective, observational study. The Lancet Respiratory Medicine (2020).
7. Gu, J. & Korteweg, C. Pathology and pathogenesis of severe acute respiratory syndrome. Am J Pathol 170, 1136-1147 (2007).
8. Goodenough, D. A. & Paul, D. L. Beyond the gap: functions of unpaired connexon channels. Nat Rev Mol Cell Biol 4, 285-294 (2003).
9. Fu, C. T., Bechberger, J. F., Ozog, M. A., Perbal, B. & Naus, C. C. CCN3 (NOV) interacts with connexin43 in C6 glioma cells: possible mechanism of connexin-mediated growth suppression. J Biol Chem 279, 36943-36950 (2004).
10. Songyang, Z. et al. Recognition of unique carboxyl-terminal motifs by distinct PDZ domains. Science 275, 73-77 (1997).
11. Rizo, J. & Gierasch, L. M. Constrained peptides: models of bioactive peptides and protein substructures. Annu Rev Biochem 61, 387-418 (1992).
12. Ghatnekar, G. S. et al. Connexin43 carboxyl-terminal peptides reduce scar progenitor and promote regenerative healing following skin wounding. Regen Med 4, 205-223 (2009).
13. Gourdie, R. G. et al. The unstoppable connexin43 carboxyl-terminus: new roles in gap junction organization and wound healing. Ann N Y Acad Sci 1080, 49-62 (2006).
14. Rhett, J. M. et al. Novel therapies for scar reduction and regenerative healing of skin wounds. Trends Biotechnol 26, 173-180 (2008).
15. Rhett, J. M., Jourdan, J. & Gourdie, R. G. Connexin 43 connexon to gap junction transition is regulated by zonula occludens-1. Mol Biol Cell 22, 1516-1528 (2011).
16. Hunter, A. W., Barker, R. J., Zhu, C. & Gourdie, R. G. Zonula occludens-1 alters connexin43 gap junction size and organization by influencing channel accretion. Mol Biol Cell 16, 5686-5698 (2005).
17. Jiang, J. et al. Interaction of alpha Carboxyl Terminus 1 Peptide With the Connexin 43 Carboxyl Terminus Preserves Left Ventricular Function After Ischemia-Reperfusion Injury. J Am Heart Assoc 8, e012385 (2019).
18. Park, D. J., Freitas, T. A., Wallick, C. J., Guyette, C. V. & Warn-Cramer, B. J. Molecular dynamics and in vitro analysis of Connexin43: A new 14-3-3 mode-1 interacting protein. Protein Sci 15, 2344-2355 (2006).
19. Scemes, E. Modulation of astrocyte P2Y1 receptors by the carboxyl terminal domain of the gap junction protein Cx43. Glia 56, 145-153 (2008).
20. Solan, J. L. & Lampe, P. D. Key connexin 43 phosphorylation events regulate the gap junction life cycle. J Membr Biol 217, 35-41 (2007).
21. Thevenin, A. F., Margraf, R. A., Fisher, C. G., Kells-Andrews, R. M. & Falk, M. M. Phosphorylation regulates connexin43/ZO-1 binding and release, an important step in gap junction turnover. Mol Biol Cell 28, 3595-3608 (2017).
22. Ek-Vitorin, J. F., King, T. J., Heyman, N. S., Lampe, P. D. & Burt, J. M. Selectivity of connexin 43 channels is regulated through protein kinase C-dependent phosphorylation. Circ Res 98, 1498-1505 (2006).
23. O'Quinn, M. P., Palatinus, J. A., Harris, B. S., Hewett, K. W. & Gourdie, R. G. A peptide mimetic of the connexin43 carboxyl terminus reduces gap junction remodeling and induced arrhythmia following ventricular injury. Circ Res 108, 704-715 (2011).
24. Obert, E. et al. Targeting the tight junction protein, zonula occludens-1, with the connexin43 mimetic peptide, aCT1, reduces VEGF-dependent RPE pathophysiology. J Mol Med (Berl) 95, 535-552 (2017).
25. Eltzschig, H. K. et al. ATP release from activated neutrophils occurs via connexin 43 and modulates adenosine-dependent endothelial cell function. Circ Res 99, 1100-1108 (2006).
26. Dosch, M. et al. Connexin-43-dependent ATP release mediates macrophage activation during sepsis. Elife 8 (2019).
27. Calder, B. W. et al. Inhibition of connexin 43 hemichannel-mediated ATP release attenuates early inflammation during the foreign body response. Tissue Eng Part A 21, 1752-1762 (2015).
28. Niessen, H., Harz, H., Bedner, P., Kramer, K. & Willecke, K. Selective permeability of different connexin channels to the second messenger inositol 1,4,5-trisphosphate. J Cell Sci 113 (Pt 8), 1365-1372 (2000).
29. Martin, P. & Parkhurst, S. M. Parallels between tissue repair and embryo morphogenesis. Development 131, 3021-3034 (2004).
30. Bruzzone, S., Guida, L., Zocchi, E., Franco, L. & De Flora, A. Connexin 43 hemi channels mediate Ca2+-regulated transmembrane NAD+fluxes in intact cells. FASEB J 15, 10-12 (2001).
31. Stout, C. E., Costantin, J. L., Naus, C. C. & Charles, A. C. Intercellular calcium signaling in astrocytes via ATP release through connexin hemichannels. J Biol Chem 277, 10482-10488 (2002).
32. Ye, Z. C., Wyeth, M. S., Baltan-Tekkok, S. & Ransom, B. R. Functional hemichannels in astrocytes: a novel mechanism of glutamate release. J Neurosci 23, 3588-3596 (2003).
33. Cherian, P. P. et al. Mechanical strain opens connexin 43 hemichannels in osteocytes: a novel mechanism for the release of prostaglandin. Mol Biol Cell 16, 3100-3106 (2005).
34. Rana, S. & Dringen, R. Gap junction hemichannel-mediated release of glutathione from cultured rat astrocytes. Neuroscience letters 415, 45-48 (2007).
35. Saez, J. C., Retamal, M. A., Basilio, D., Bukauskas, F. F. & Bennett, M. V. Connexin-based gap junction hemichannels: gating mechanisms. Biochimica et biophysica acta 1711, 215-224 (2005).
36. Evans, W. H. & Boitano, S. Connexin mimetic peptides: specific inhibitors of gap-junctional intercellular communication. Biochemical Society transactions 29, 606-612 (2001).
37. Ramachandran, S., Xie, L. H., John, S. A., Subramaniam, S. & Lal, R. A novel role for connexin hemichannel in oxidative stress and smoking-induced cell injury. PLoS One 2, e712 (2007).
38. Rhett, J. M., Fann, S. A. & Yost, M. J. Purinergic Signaling in Early Inflammatory Events of the Foreign Body Response: Modulating Extracellular ATP as an Enabling Technology for Engineered Implants and Tissues. Tissue engineering. Part B, Reviews (2014).
39. Retamal, M. A., Cortes, C. J., Reuss, L., Bennett, M. V. & Saez, J. C. S-nitrosylation and permeation through connexin 43 hemichannels in astrocytes: induction by oxidant stress and reversal by reducing agents. Proceedings of the National Academy of Sciences of the United States of America 103, 4475-4480 (2006).
40. Retamal, M. A., Schalper, K. A., Shoji, K. F., Bennett, M. V. & Saez, J. C. Opening of connexin 43 hemichannels is increased by lowering intracellular redox potential. Proc Natl Acad Sci USA 104, 8322-8327 (2007).
41. Overgaard, C. E., Daugherty, B. L., Mitchell, L. A. & Koval, M. Claudins: control of barrier function and regulation in response to oxidant stress. Antioxid Redox Signal 15, 1179-1193 (2011).
42. Umeda, K. et al. ZO-1 and ZO-2 independently determine where claudins are polymerized in tight-junction strand formation. Cell 126, 741-754 (2006).
43. Cummins, P. M. Occludin: one protein, many forms. Mol Cell Biol 32, 242-250 (2012).
44. Tornavaca, O. et al. ZO-1 controls endothelial adherens junctions, cell-cell tension, angiogenesis, and barrier formation. J Cell Biol 208, 821-838 (2015).
45. Palatinus, J. A. et al. ZO-1 determines adherens and gap junction localization at intercalated disks. Am J Physiol Heart Circ Physiol 300, H583-594 (2011).
46. Spadaro, D. et al. Tension-Dependent Stretching Activates ZO-1 to Control the Junctional Localization of Its Interactors. Curr Biol 27, 3783-3795 e3788 (2017).
47. Obert, E. et al. Targeting the tight junction protein, zonula occludens-1, with the connexin43 mimetic peptide, alphaCT1, reduces VEGF-dependent RPE pathophysiology. J Mol Med (Berl) 95, 535-552 (2017).
48. Montgomery, J. et al. The connexin 43 carboxyl terminal mimetic peptide alphaCT1 prompts differentiation of a collagen scar matrix in humans resembling unwounded skin. FASEB J 35, e21762 (2021).
49. Soder, B. L. et al. The connexin43 carboxyl-terminal peptide ACT1 modulates the biological response to silicone implants. Plast Reconstr Surg 123, 1440-1451 (2009).
50. Xu, H. et al. Blocking connexin 43 and its promotion of ATP release from renal tubular epithelial cells ameliorates renal fibrosis. Cell Death Dis 13, 511 (2022).
51. Ghatnekar, G., Grek, C., Armstrong, D. G., Desai, S. C. & Gourdie, R. The Effect of a Connexin43-based peptide on the Healing of Chronic Venous Leg Ulcers: A Multicenter, Randomized Trial. J Invest Dermatol (2014).
52. Ghatnekar, G. S., Grek, C. L., Armstrong, D. G., Desai, S. C. & Gourdie, R. G. The effect of a connexin43-based Peptide on the healing of chronic venous leg ulcers: a multicenter, randomized trial. J Invest Dermatol 135, 289-298 (2015).
53. Grek, C. et al. A Multicenter, Randomized, Controlled Trial Evaluating a Connexin43-Mimetic Peptide in Cutaneous Scarring. Journal of Investigative Dermatology (2016).
54. McAuliffe, J. et al. Replication of SARS coronavirus administered into the respiratory tract of African Green, rhesus and cynomolgus monkeys. Virology 330, 8-15 (2004).
55. Clay, C. et al. Primary severe acute respiratory syndrome coronavirus infection limits replication but not lung inflammation upon homologous rechallenge. J Virol 86, 4234-4244 (2012).
56. Clay, C. C. et al. Severe acute respiratory syndrome-coronavirus infection in aged nonhuman primates is associated with modulated pulmonary and systemic immune responses. Immun Ageing 11, 4 (2014).
57. Imai, M. et al. Syrian hamsters as a small animal model for SARS-CoV-2 infection and countermeasure development. Proc Natl Acad Sci USA 117, 16587-16595 (2020).
58. Alexander, D. J. et al. Association of Inhalation Toxicologists (AIT) working party recommendation for standard delivered dose calculation and expression in non-clinical aerosol inhalation toxicology studies with pharmaceuticals. Inhal Toxicol 20, 1179-1189 (2008).
59. Tepper, J. S. et al. Symposium Summary: “Breathe In, Breathe Out, Its Easy: What You Need to Know About Developing Inhaled Drugs”. Int J Toxicol 35, 376-392 (2016).

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### FURTHER NUMBERED EMBODIMENTS OF THE DISCLOSURE

Other subject matter contemplated by the present disclosure is set out in the following numbered embodiments:

1. A method of treating or preventing a complication of a respiratory viral disease in a subject, comprising administering to the subject a composition comprising an isolated polypeptide comprising the carboxy terminal-most 4 to 30 contiguous amino acids of an alpha Connexin and administering to the subject a composition comprising an antiviral agent.

2. The method of embodiment 1, wherein the respiratory viral disease is caused by SARS-CoV-2.

3. The method of embodiment 1, wherein the respiratory viral disease is caused by an Influenza A, B, or C virus.

4. The method of embodiment 2, wherein the respiratory viral disease is SARS-CoV-2-induced ARDS and/or ALI.

5. The method of embodiment 2, wherein the respiratory viral disease is Respiratory Syncytial Virus-induced ARDS and/or ALI.

6. The method of any one of embodiments 1-5, wherein the polypeptide comprises the amino acid sequence selected from the group consisting of SEQ ID NO: 1, SEQ ID NO: 2, SEQ ID NO: 3, SEQ ID NO: 4, and SEQ ID NO: 5.

7. The method of embodiment 6, wherein the polypeptide comprises the amino sequence of SEQ ID NO: 2.

8. The method of any one of embodiments 1-7, wherein the polypeptide further comprises a cellular internalization sequence.

9. The method of embodiment 8, wherein the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

10. The method of embodiment 9, wherein the cellular internalization sequence is Antennapedia, and wherein the sequence comprises the amino acid sequence of SEQ ID NO:7.

11. The method of any one of embodiments 1-10, wherein the polypeptide comprises the amino acid sequence selected from the group consisting of SEQ ID NO:8, SEQ ID NO: 9, SEQ ID NO: 10, SEQ ID NO: 11, and SEQ ID NO: 12.

12. The method of embodiment 11, wherein the polypeptide comprises the amino acid sequence of SEQ ID NO:9.

13. The method of any one of embodiments 1-12, wherein the composition comprising the polypeptide is administered to the subject intravenously, parenterally, intranasally, intratracheally, by inhalant, or by topical intranasal administration.

14. The method of any one of embodiments 1-13, wherein the composition comprising the polypeptide is administered to the subject by aerosolized delivery.

15. The method of any one of embodiments 1-14, wherein the composition comprising the polypeptide is administered via an inhaler or a nebulizer.

16. The method of any one of embodiments 1-15, wherein the composition comprising the polypeptide is administered to the subject in a drug loaded microcarrier formulation.

17. The method of embodiment 16, wherein the drug loaded microcarrier formulation comprises nanoparticles or exosomes.

18. The method of any one of the above embodiments, wherein the composition comprising the antiviral agent is administered intravenously.

19. The method of embodiments 1-17, wherein the composition comprising the antiviral agent is administered orally.

20. The method of any one of embodiments 1-19, wherein the composition comprising the polypeptide and/or the composition comprising the antiviral agent is/are administered to the subject at the onset of infection.

21. The method of any one of embodiments 1-19, wherein the composition comprising the polypeptide and/or the composition comprising the antiviral agent is/are administered to the subject prior to the onset of symptoms of the respiratory viral disease.

22. The method of any one of embodiments 1-19, wherein the composition comprising the polypeptide and/or the composition comprising the antiviral agent is/are administered to the subject after onset of symptoms of the respiratory viral disease.

23. The method of any one of the above embodiments, wherein the method prevents lung injury caused by the respiratory viral disease.

24. The method of any one of embodiments 1-22, wherein the method limits the progression of lung injury caused by the respiratory viral disease.

25. The method of any one of the above embodiments, wherein the method maintains lung function after the onset of the respiratory viral disease.

26. The method of any one of the above embodiments, wherein the method reduces lung inflammation as compared to administration of the polypeptide or the anti-viral agent alone.

27. The method of any one of the above embodiments, wherein the method reduces pulmonary edema as compared to administration of the polypeptide or the anti-viral agent alone.

28. The method of any one of the above embodiments, wherein the method reduces alveolar hemorrhage as compared to administration of the polypeptide or the anti-viral agent alone.

29. The method of any one of the above embodiments, wherein the anti-viral agent prevents viral replication.

30. The method of any one of the above embodiments, wherein the anti-viral agent is an inhibitor of a viral RNA-dependent, RNA polymerase.

31. The method of any one of the above embodiments, wherein the anti-viral agent is a prodrug or a protide.

32. The method of any one of the above embodiments, wherein the anti-viral agent is a nucleoside analog.

33. The method of embodiment 32, wherein the nucleoside analog is an adenosine analog.

34. The method of embodiment 32 or 33, wherein the nucleoside analog is remdesivir.

35. The method of embodiment 34, wherein the nucleoside analog is an active metabolite thereof.

36. The method of any one of embodiments 32-35, wherein the nucleoside analog is a derivative of remdesivir.

37. The method of embodiment 36, wherein the derivative of remdesivir is selected from the group consisting of ATV006, GS-621763 and VV116.

38. A method for treating or preventing a respiratory disease or disorder in a subject in need thereof, the method comprising administering to the subject a composition comprising an isolated polypeptide comprising the carboxy terminal-most 4 to 30 contiguous amino acids of an alpha Connexin; and administering to the subject a composition comprising an antiviral agent.

39. The method of embodiment 38, wherein the respiratory disease or disorder is pneumonia, acute respiratory distress syndrome (ARDS), alcoholic lung syndrome, acute lung injury, chronic obstructive pulmonary disease (COPD), or pulmonary fibrosis.

40. The method of embodiment 39, wherein the pulmonary fibrosis is idiopathic pulmonary fibrosis (IPF).

41. The method of any one of embodiments 38-40, wherein the polypeptide comprises the amino acid sequence selected from the group consisting of SEQ ID NO: 1, SEQ ID NO: 2, SEQ ID NO: 3, SEQ ID NO: 4, and SEQ ID NO: 5.

42. The method of embodiment 41, wherein the polypeptide comprises the amino sequence of SEQ ID NO: 2.

43. The method of any one of embodiments 38-42, wherein the polypeptide further comprises a cellular internalization sequence.

44. The method of embodiment 43, wherein the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

45. The method of embodiment 44, wherein the cellular internalization sequence is Antennapedia.

46. The method of embodiment 45, wherein the Antennapedia sequence comprises the amino acid sequence of SEQ ID NO:7.

47. The method of any one of embodiments 38-46, wherein the polypeptide comprises the amino acid sequence selected from the group consisting of SEQ ID NO:8, SEQ ID NO: 9, SEQ ID NO: 10, SEQ ID NO: 11, and SEQ ID NO: 12.

48. The method of embodiment 47, wherein the polypeptide comprises the amino acid sequence of SEQ ID NO:9.

49. The method of any one of embodiments 38-48, wherein the composition comprising the polypeptide is administered to the subject intravenously, parenterally, intranasally, intratracheally, by inhalant, or by topical intranasal administration.

50. The method of any one of embodiments 38-49, wherein the composition comprising the polypeptide is administered to the subject by aerosolized delivery.

51. The method of any one of embodiments 38-49, wherein the composition comprising the polypeptide is administered via an inhaler or a nebulizer.

52. The method of any one of embodiments 38-51, wherein the composition comprising the polypeptide is administered to the subject in a drug loaded microcarrier formulation.

53. The method of embodiment 52, wherein the drug loaded microcarrier formulation comprises nanoparticles or exosomes.

54. The method of any one of embodiments 38-53, wherein the composition comprising the anti-viral agent is administered intravenously.

55. The method of embodiments 38-54, wherein the composition comprising the antiviral agent is administered orally.

56. The method of embodiments 38-55, wherein the anti-viral agent prevents viral replication.

57. The method of embodiments 38-56, wherein the anti-viral agent is an inhibitor of a viral RNA-dependent, RNA polymerase.

58. The method of embodiments 38-57, wherein the anti-viral agent is a prodrug or a protide.

59. The method of embodiments 38-58, wherein the anti-viral agent is a nucleoside analog.

60. The method of embodiment 59, wherein the nucleoside analog is an adenosine analog.

61. The method of embodiment 59 or 60, wherein the nucleoside analog is remdesivir.

62. The method of embodiment 61, wherein the nucleoside analog is an active metabolite thereof.

63. The method of any one of embodiments 59-62, wherein the nucleoside analog is a derivative of remdesivir.

64. The method of embodiment 63, wherein the derivative of remdesivir is selected from the group consisting of ATV006, GS-621763 and VV116.

65. A composition for use in treating or preventing a complication of a respiratory viral disease in a subject, wherein the composition comprises a polypeptide comprising the carboxy terminal-most 4 to 30 contiguous amino acids of an alpha Connexin and an anti-viral agent.

66. A composition for use in treating or preventing a respiratory disorder in a subject, wherein the composition comprises a polypeptide comprising the carboxy terminal-most 4 to 40 contiguous amino acids of an alpha Connexin and an anti-viral agent.

67. The composition of embodiment 65 or 66, wherein the anti-viral agent prevents viral replication.

68. The composition of embodiments 65-67, wherein the anti-viral agent is an inhibitor of a viral RNA-dependent, RNA polymerase.

69. The composition of embodiments 65-68, wherein the anti-viral agent is a prodrug or a protide.

70. The composition of embodiments 65-69, wherein the anti-viral agent is a nucleoside analog.

71. The composition of embodiment 70, wherein the nucleoside analog is an adenosine analog.

72. The composition of embodiment 70 or 71, wherein the nucleoside analog is remdesivir.

73. The composition of embodiment 72, wherein the nucleoside analog is an active metabolite thereof.

74. The composition of any one of embodiments 70-73, wherein the nucleoside analog is a derivative of remdesivir.

75. The composition of embodiment 74, wherein the derivative of remdesivir is selected from the group consisting of ATV006, GS-621763 and VV116.

1. A method of treating or preventing a complication of a respiratory viral disease in a subject, comprising administering to the subject a composition comprising an isolated polypeptide comprising the carboxy terminal-most 4 to 30 contiguous amino acids of an alpha Connexin and administering to the subject a composition comprising an antiviral agent.

2. The method of claim 1, wherein the respiratory viral disease is caused by SARS-CoV-2.

3. The method of claim 1, wherein the respiratory viral disease is caused by an Influenza A, B, or C virus.

4. The method of claim 2, wherein the respiratory viral disease is SARS-CoV-2-induced ARDS and/or ALI.

5. The method of claim 2, wherein the respiratory viral disease is Respiratory Syncytial Virus-induced ARDS and/or ALI.

6. The method of claim 1, wherein the polypeptide comprises the amino acid sequence selected from the group consisting of SEQ ID NO: 1, SEQ ID NO: 2, SEQ ID NO: 3, SEQ ID NO: 4, and SEQ ID NO: 5.

7. The method of claim 6, wherein the polypeptide comprises the amino sequence of SEQ ID NO: 2.

8. The method of claim 1, wherein the polypeptide further comprises a cellular internalization sequence.

9. The method of claim 8, wherein the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

10. The method of claim 9, wherein the cellular internalization sequence is Antennapedia, and wherein the sequence comprises the amino acid sequence of SEQ ID NO:7.

11. The method of claim 1, wherein the polypeptide comprises the amino acid sequence selected from the group consisting of SEQ ID NO:8, SEQ ID NO:9, SEQ ID NO: 10, SEQ ID NO: 11, and SEQ ID NO: 12.

12. The method of claim 11, wherein the polypeptide comprises the amino acid sequence of SEQ ID NO:9.

13. The method of claim 1, wherein the composition comprising the polypeptide is administered to the subject intravenously, parenterally, intranasally, intratracheally, by inhalant, or by topical intranasal administration.

14. The method of claim 1, wherein the composition comprising the polypeptide is administered to the subject by aerosolized delivery.

15. The method of claim 1, wherein the composition comprising the polypeptide is administered via an inhaler or a nebulizer.

16. The method of claim 1, wherein the composition comprising the polypeptide is administered to the subject in a drug loaded microcarrier formulation.

17. The method of claim 16, wherein the drug loaded microcarrier formulation comprises nanoparticles or exosomes.

18. The method of claim 1, wherein the composition comprising the antiviral agent is administered intravenously.

19. The method of claim 1, wherein the composition comprising the antiviral agent is administered orally.

20. The method of claim 1, wherein the composition comprising the polypeptide and/or the composition comprising the antiviral agent is/are administered to the subject at the onset of infection.

21. The method of claim 1, wherein the composition comprising the polypeptide and/or the composition comprising the antiviral agent is/are administered to the subject prior to the onset of symptoms of the respiratory viral disease.

22. The method of claim 1, wherein the composition comprising the polypeptide and/or the composition comprising the antiviral agent is/are administered to the subject after onset of symptoms of the respiratory viral disease.

23. The method of claim 1, wherein the method prevents lung injury caused by the respiratory viral disease.

24. The method of claim 1, wherein the method limits the progression of lung injury caused by the respiratory viral disease.

25. The method of claim 1, wherein the method maintains lung function after the onset of the respiratory viral disease.

26. The method of claim 1, wherein the method reduces lung inflammation as compared to administration of the polypeptide or the anti-viral agent alone.

27. The method of claim 1, wherein the method reduces pulmonary edema as compared to administration of the polypeptide or the anti-viral agent alone.

28. The method of claim 1, wherein the method reduces alveolar hemorrhage as compared to administration of the polypeptide or the anti-viral agent alone.

29. The method of claim 1, wherein the anti-viral agent prevents viral replication.

30. The method of claim 1, wherein the anti-viral agent is an inhibitor of a viral RNA-dependent, RNA polymerase.

31. The method of claim 1, wherein the anti-viral agent is a prodrug or a protide.

32. The method of claim 1, wherein the anti-viral agent is a nucleoside analog.

33. The method of claim 32, wherein the nucleoside analog is an adenosine analog.

34. The method of claim 32 or 33, wherein the nucleoside analog is remdesivir.

35. The method of claim 34, wherein the nucleoside analog is an active metabolite thereof.

36. The method of claim 32, wherein the nucleoside analog is a derivative of remdesivir.

37. The method of claim 36, wherein the derivative of remdesivir is selected from the group consisting of ATV006, GS-621763 and VV116.

38. A method for treating or preventing a respiratory disease or disorder in a subject in need thereof, the method comprising administering to the subject a composition comprising an isolated polypeptide comprising the carboxy terminal-most 4 to 30 contiguous amino acids of an alpha Connexin; and administering to the subject a composition comprising an antiviral agent.

39. The method of claim 38, wherein the respiratory disease or disorder is pneumonia, acute respiratory distress syndrome (ARDS), alcoholic lung syndrome, acute lung injury, chronic obstructive pulmonary disease (COPD), or pulmonary fibrosis.

40. The method of claim 39, wherein the pulmonary fibrosis is idiopathic pulmonary fibrosis (IPF).

41. The method of claim 38, wherein the polypeptide comprises the amino acid sequence selected from the group consisting of SEQ ID NO: 1, SEQ ID NO: 2, SEQ ID NO: 3, SEQ ID NO: 4, and SEQ ID NO: 5.

42. The method of claim 41, wherein the polypeptide comprises the amino sequence of SEQ ID NO: 2.

43. The method of claim 38, wherein the polypeptide further comprises a cellular internalization sequence.

44. The method of claim 43, wherein the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

45. The method of claim 44, wherein the cellular internalization sequence is Antennapedia.

46. The method of claim 45, wherein the Antennapedia sequence comprises the amino acid sequence of SEQ ID NO:7.

47. The method of claim 38, wherein the polypeptide comprises the amino acid sequence selected from the group consisting of SEQ ID NO:8, SEQ ID NO:9, SEQ ID NO:10, SEQ ID NO: 11, and SEQ ID NO:12.

48. The method of claim 47, wherein the polypeptide comprises the amino acid sequence of SEQ ID NO:9.

49. The method of claim 38, wherein the composition comprising the polypeptide is administered to the subject intravenously, parenterally, intranasally, intratracheally, by inhalant, or by topical intranasal administration.

50. The method of claim 38, wherein the composition comprising the polypeptide is administered to the subject by aerosolized delivery.

51. The method of claim 38, wherein the composition comprising the polypeptide is administered via an inhaler or a nebulizer.

52. The method of claim 38, wherein the composition comprising the polypeptide is administered to the subject in a drug loaded microcarrier formulation.

53. The method of claim 52, wherein the drug loaded microcarrier formulation comprises nanoparticles or exosomes.

54. The method of claim 38, wherein the composition comprising the anti-viral agent is administered intravenously.

55. The method of claim 38, wherein the composition comprising the antiviral agent is administered orally.

56. The method of claim 38, wherein the anti-viral agent prevents viral replication.

57. The method of claim 38, wherein the anti-viral agent is an inhibitor of a viral RNA-dependent, RNA polymerase.

58. The method of claim 38, wherein the anti-viral agent is a prodrug or a protide.

59. The method of claim 38, wherein the anti-viral agent is a nucleoside analog.

60. The method of claim 59, wherein the nucleoside analog is an adenosine analog.

61. The method of claim 59 or 60, wherein the nucleoside analog is remdesivir.

62. The method of claim 61, wherein the nucleoside analog is an active metabolite thereof.

63. The method of claim 59, wherein the nucleoside analog is a derivative of remdesivir.

64. The method of claim 63, wherein the derivative of remdesivir is selected from the group consisting of ATV006, GS-621763 and VV116.

65. A composition for use in treating or preventing a complication of a respiratory viral disease in a subject, wherein the composition comprises a polypeptide comprising the carboxy terminal-most 4 to 30 contiguous amino acids of an alpha Connexin and an anti-viral agent.

66. A composition for use in treating or preventing a respiratory disorder in a subject, wherein the composition comprises a polypeptide comprising the carboxy terminal-most 4 to 40 contiguous amino acids of an alpha Connexin and an anti-viral agent.

67. The composition of claim 65 or 66, wherein the anti-viral agent prevents viral replication.

68. The composition of claim 65 or 66, wherein the anti-viral agent is an inhibitor of a viral RNA-dependent, RNA polymerase.

69. The composition of claim 65 or 66, wherein the anti-viral agent is a prodrug or a protide.

70. The composition of claim 65 or 66, wherein the anti-viral agent is a nucleoside analog.

71. The composition of claim 70, wherein the nucleoside analog is an adenosine analog.

72. The composition of claim 70, wherein the nucleoside analog is remdesivir.

73. The composition of claim 72, wherein the nucleoside analog is an active metabolite thereof.

74. The composition of claim 70, wherein the nucleoside analog is a derivative of remdesivir.

75. The composition of claim 74, wherein the derivative of remdesivir is selected from the group consisting of ATV006, GS-621763 and VV116.
